(19) United States
(12) Patent Application Publication
(10) Pub. No.: US 2020/0149038 A1
(43) Pub. Date: May 14, 2020

(54) METHODS AND COMPOSITIONS RELATING TO HOMOLOGY-DIRECTED REPAIR

(71) Applicant: CHILDREN'S MEDICAL CENTER CORPORATION, Boston, MA (US)

(72) Inventors: Derrick J. ROSSI, Boston, MA (US); Bruna PAULSEN, Boston, MA (US); Pankaj K. MANDAL, Cambridge, MA (US); Wataru EBINA, Boston, MA (US); Paula GUTIERREZ-MARTINEZ, Brookline, MA (US)

(73) Assignee: CHILDREN'S MEDICAL CENTER CORPORATION, Boston, MA (US)

(21) Appl. No.: 16/088,550

(22) PCT Filed: Mar. 28, 2017
(86) PCT No.: PCT/US17/24548

§ 371 Date: Sep. 26, 2018

Related U.S. Application Data
(60) Provisional application No. 62/316,797, filed on Apr. 1, 2016.

(51) Int. Cl. C12N 15/11; C12N 9/22

(52) U.S. Cl. CPC C12N 15/11; C12N 2310/20; C12N 2800/80; C12N 9/22

(57) ABSTRACT

The methods and compositions described herein relate to improvements in the efficiency and/or accuracy of targeted alterations to a nucleic acid sequence, e.g, gene editing technologies, by creating nick or DSB in a target nucleic in the presence of template molecule, an inhibitor of NHEJ and an agonist of HDR. In contrast to earlier technologies, these methods are not specific to each template and/or target sequence while retaining specificity of the editing itself.

### CROSS-REFERENCE TO RELATED APPLICATIONS

This application claims benefit under 35 U.S.C. § 119(e) of U.S. Provisional Application No. 62/316,797 filed Apr. 1, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Mar. 22, 2017, is named 701039-085691-PCT_SL.txt and is 82,008 bytes in size.

### TECHNICAL FIELD

The technology described herein relates to methods and compositions for altering a nucleic acid sequence, e.g., for gene editing applications.

### BACKGROUND

The CRISPR/Cas9 system is a technology that permits users to create cuts in the DNA strands of a cell's genome at any desired location, e.g., in a gene. Without further input from the user, the cell will attempt to repair these cuts predominantly through a mechanism called non-homologous end joining (NHEJ). NHEJ has a high error rate, and so these repair attempts are likely to alter the target gene such that it no longer encodes a functional protein.

Particularly for clinical applications, it is often desired to correct errors (mutations) in a gene, not to create new errors. CRISPR/Cas9 makes such corrections possible. When a DNA template, e.g. a DNA molecule with the “correct” sequence is provided to the cell with the CRISPR/Cas9 system, the cell can attempt to use the template to fix the cut made by the CRISPR/Cas9 system itself. Such repairs are performed by the cell's homology-directed repair (HDR) pathway. Unfortunately, as compared to NHEJ, HDR-mediated repair is relatively infrequent since, e.g., it is only engaged during specific phases (e.g., S/G2 phase) of the cell cycle whereas NHEJ is active through the cell cycle.

### SUMMARY

Attempts to improve the efficiency of HDR, e.g., by arresting the cell cycle at points which favor HDR has been of limited utility. The gains in efficiency accomplished by existing methods are minimal and often must be tediously optimized for each new template DNA and/or target sequence. As described herein, the inventors have discovered that simultaneous inhibition of certain selected proteins involved in NHEJ and promotion of certain selected proteins involved in HDR provides striking and surprising increases in the rate of HDR as opposed to NHEJ. These gains in HDR frequency are unprecedented in magnitude and appear to be universal across a number of template/target combinations. Furthermore, no loss in specificity of DNA editing is observed. Accordingly, described herein are methods relating to altering a target sequence of a target nucleic acid molecule, e.g., in the presence of an inhibitor of NHEJ and an agonist of HDR.

In one aspect of any of the embodiments, described herein is a method of altering a target sequence of a target nucleic acid molecule, the method comprising contacting the target nucleic acid molecule with: a) a nuclease; b) at least one inhibitor of non-homologous end joining (NHEJ); c) at least one agonist of homology-directed repair (HDR); and d) a template nucleic acid. In some embodiments of any of the aspects, the inhibitor of NHEJ is selected from the group consisting of: an inhibitor of Ku70; an inhibitor of Ku80; and an inhibitor of 53BP1. In some embodiments of any of the aspects, the agonist of HDR is selected from the group consisting of: an agonist of RAD52 and an agonist of RAD51. In some embodiments of any of the aspects, the agonist of HDR is selected from the group consisting of: an agonist of RAD52; an agonist of RAD51; and an agonist of BLM. In some embodiments of any of the aspects, the inhibitor of NHEJ is an inhibitor of 53BP1 and the agonist of HDR is an agonist of Rad52.

In one aspect of any of the embodiments, described herein is a method of altering the sequence of a target nucleic acid molecule, the method comprising contacting the target nucleic acid molecule with: a) a nuclease; b) a template nucleic acid; and c) at least one inhibitor of 53BP1 and/or at least one agonist of RAD52. In some embodiments of any of the aspects, the target nucleic acid molecule is contacted with at least one inhibitor of 53BP1 and at least one agonist of RAD52.

In one aspect of any of the embodiments, described herein is a method of altering the sequence of a target nucleic acid molecule, the method comprising contacting the target nucleic acid molecule with: a) a nuclease; and b) at least one agonist of RAD52. In some embodiments of any of the aspects, the target nucleic acid molecule is contacted with an inhibitor of 53BP1.

In some embodiments of any of the aspects, the agonist of Rad52 is ectopic Rad52 polypeptide or a constitutively active RAD52 polypeptide. In some embodiments of any of the aspects, the agonist of RAD51 is ectopic RAD51 polypeptide or a constitutively active RAD51 polypeptide. In some embodiments of any of the aspects, the agonist of RAD51 is constitutively active RAD51 polypeptide. In some embodiments of any of the aspects, the agonist of BLM is ectopic BLM polypeptide. In some embodiments of any of the aspects, the target nucleic acid is contacted with the ectopic polypeptide by delivering a polypeptide to the target nucleic acid. In some embodiments of any of the aspects, the target nucleic acid is contacted with the ectopic polypeptide by delivering a nucleic acid encoding the polypeptide to the target nucleic acid.

In some embodiments of any of the aspects, the inhibitor of NHEJ is an inhibitor of Lig4. In some embodiments of any of the aspects, the inhibitor of Lig4 is SCR7. In some embodiments of any of the aspects, the target nucleic acid molecule is contacted with at least one agonist of HDR selected from E1B55K and E4orf6. In some embodiments of any of the aspects, the inhibitor of Ku70 is an inhibitory nucleic acid. In some embodiments of any of the aspects, the inhibitor of Ku80 is an inhibitory nucleic acid. In some embodiments of any of the aspects, the inhibitor of 53BP1 is an inhibitory nucleic acid or a dominant-negative 53BP1 (dn53BP1) polypeptide. In some embodiments of any of the aspects, the target nucleic acid is contacted with the dn53BP1 polypeptide by delivering a polypeptide to the target nucleic acid. In some embodiments of any of the aspects, the target nucleic acid is contacted with the dn53BP1 polypeptide by delivering a nucleic acid encoding the polypeptide to the target nucleic acid.

In some embodiments of any of the aspects, the nucleic acid encoding a polypeptide is an mRNA. In some embodiments of any of the aspects, the mRNA is a modified mRNA.

In some embodiments of any of the aspects, the nuclease is a programmable nuclease. In some embodiments of any of the aspects, the programmable nuclease is selected from the group consisting of: Cas9; a Cas9 nickase mutant; TALEN; ZFNs; Cpf1; and SaCas9. In some embodiments of any of the aspects, the programmable nuclease is Cas9. In some embodiments of any of the aspects, the method further comprises contacting the target nucleic acid molecule with a guide RNA that can hybridize to a portion of the target nucleic acid molecule. In some embodiments of any of the aspects, the nuclease is a Cas9 or Cas9-derived nuclease and the method further comprises contacting the target nucleic acid molecule with a guide RNA that can hybridize to a portion of the target nucleic acid molecule. In some embodiments of any of the aspects, the nuclease is a meganuclease. In some embodiments of any of the aspects, the template nucleic acid is selected from the group consisting of: a single-stranded DNA molecule; a double-stranded DNA molecule; a DNA/RNA hybrid molecule; and a DNA/modRNA hybrid molecule.

In some embodiments of any of the aspects, the contacting step occurs in a cell. In some embodiments of any of the aspects, the cell is a eukaryotic cell. In some embodiments of any of the aspects, the cell is a mammalian cell. In some embodiments of any of the aspects, the cell is a human cell. In some embodiments of any of the aspects, the cell is a stem cell or iPSC. In some embodiments of any of the aspects, the cell is a hematopoietic cell, hematopoietic stem cell, or hematopoietic progenitor cell.

In some embodiments of any of the aspects, the target nucleic acid molecule is a chromosome. In some embodiments of any of the aspects, the target sequence is located in the genomic DNA or the mitochondrial DNA. In some embodiments of any of the aspects, the target sequence is located at a locus, a coding gene sequence, or a regulatory region. In some embodiments of any of the aspects, the target sequence is comprised by the HBB gene. In some embodiments of any of the aspects, the target sequence is comprised by the ADA gene; IL-2Rγ gene; PNP gene; RAG-1 gene; RAG-2 gene; JAK3 gene; AK2 gene; or DCLRE1C gene.

In some embodiments of any of the aspects, the on-target or off-target cutting specificity of Cas9 activity is not altered by inclusion of the at least one inhibitor of NHEJ and/or at least one agonist of HDR.

In some embodiments of any of the aspects, the method further comprises contacting the cell with a cell cycle modulator. In some embodiments of any of the aspects, the cell cycle modulator increases the proportion of cells in late S or G2 phase. In some embodiments of any of the aspects, the method further comprises contacting the cell with at least one factor that increases the survival, maintenance, and/or expansion of hematopoietic stem and progenitor cells.

In some embodiments of any of the aspects, the frequency of HDR is increased at least 1.25 fold relative to the frequency of HDR in the absence of the at least one inhibitor of non-homologous end joining (NHEJ) and the at least one agonist of homology-directed repair (HDR).

In one aspect of any of the embodiments, described herein is a composition comprising a) at least one inhibitor of non-homologous end joining (NHEJ); and/or b) at least one agonist of homology-directed repair (HDR). In one aspect of any of the embodiments, described herein is a kit comprising: a) a cell comprising a target nucleic acid molecule and/or a nuclease; b) at least one inhibitor of non-homologous end joining (NHEJ); and/or c) at least one agonist of homology-directed repair (HDR). In some embodiments of any of the aspects, the inhibitor and/or agonist are expressed from a nucleic acid molecule comprised by the cell.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the ratio of the Cas9 nuclease:gRNA is 1:4 or greater. In some embodiments of any of the aspects, the ratio of the Cas9 nuclease:gRNA is 1:4 to 8:1. In some embodiments of any of the aspects, the concentration of the Cas9 nuclease does not exceed 200 ng/5000 cells. In some embodiments of any of the aspects, the concentration of the gRNA does not exceed 100 ng/5000 cells. In some embodiments of any of the aspects, the concentration of the Cas9 nuclease does not exceed 200 ng/5000 cells and the concentration of the gRNA does not exceed 100 ng/5000 cells. In some embodiments of any of the aspects, the concentration of the template nucleic acid is 2 pmol/5000 cells or greater. In some embodiments of any of the aspects, the concentration of the template nucleic acid is 20 pmol/5000 or less. In some embodiments of any of the aspects, the concentration of the template nucleic acid is from 2 pmol/5000 cells to 20 pmol/5000 cells. In some embodiments of any of the aspects, the concentration of the template nucleic acid is from 2 pmol/5000 cells to 12 pmol/5000 cells. In some embodiments of any of the aspects, the concentration of the template nucleic acid is from 4 pmol/5000 cells to 20 pmol/5000 cells. In some embodiments of any of the aspects, the concentration of the template nucleic acid is from 4 pmol/5000 cells to 12 pmol/5000 cells. In some embodiments of any of the aspects, the template nucleic acid has a portion with homology to the target nucleic acid molecule that is greater than 100 bp in length. In some embodiments of any of the aspects, the template nucleic acid has a portion with homology to the target nucleic acid molecule that is 142 bp or greater in length. In some embodiments of any of the aspects, the template nucleic acid has a portion with homology to the target nucleic acid molecule that is 184 bp or greater in length.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the concentration of the template nucleic acid is 2 pmol/5000 cells or greater. In some embodiments of any of the aspects, the concentration of the template nucleic acid is from 2 pmol/5000 cells to 20 pmol/5000 cells. In some embodiments of any of the aspects, the concentration of the template nucleic acid is from 2 pmol/5000 cells to 12 pmol/5000 cells. In some embodiments of any of the aspects, the concentration of the template nucleic acid is from 4 pmol/5000 cells to 20 pmol/5000 cells. In some embodiments of any of the aspects, the concentration of the template nucleic acid is from 4 pmol/5000 cells to 12 pmol/5000 cells.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the template nucleic acid has a portion with homology to the target nucleic acid molecule that is greater than 100 bp in length. In some embodiments of any of the aspects, the template nucleic acid has a portion with homology to the target nucleic acid molecule that is 142 bp or greater in length. In some embodiments of any of the aspects, the template nucleic acid has a portion with homology to the target nucleic acid molecule that is 184 bp or greater in length.

In some embodiments of any of the aspects, the template nucleic acid has a portion with homology to the sense strand of the target nucleic acid molecule.

### BRIEF DESCRIPTION OF THE DRAWINGS

FIGS. 1A-1I demonstrate that ectopic expression of Rad52 and dominant negative 53BP1 (dn53BP1) increases HDR frequency. FIG. 1A depicts a schematic representation of major candidate genes involved in determining the DNA damage repair pathway choice. Factors suppressing NHEJ and promoting HDR are shown. FIG. 1B depicts a graph of siRNA-mediated knock-down of Ku70 and Ku80. Bar graph represents % HDR estimated by GFP+ cells for each conditions. FIG. 1C depicts a graph demonstrating that improving the HDR efficiency through ectopic expression of HDR promoting factors (Rad51, Rad52, BLM, EXO, dn53BP1) and respective phospho-mutants (EXO1^{S714E}, RAD51^{S309E}, RAD52^{Y104E} all denoted with an asterisks). 4 different plasmid concentrations of each factor are shown. FIG. 1D depicts a bar graph showing percentage of GFP positive cells after co-transfection of Cas9, gRNA and donor combined to different compositions of the NHEJ blockers (perpendicular slashes) or HDR enhancers (grey) or all the candidates (slashes). FIG. 1E depicts a bar graph showing various combinations of factors to improve HDR. The combinations that significantly improve HDR efficiency are marked with (*). Highly efficient combinations are marked by (#). Combination of all the candidate factors reveals 9 conditions in which the efficiency of gene correction is the same (#). FIG. 1F depicts histograms of GFP expression in different conditions. Significance was calculated using the One-way ANOVA (**p<0.01, ***p 0.001, ns: not significant). The bars represent mean values±s.e.m. Scale bars: 100 μm. FIGS. 1G-1H depict representative FACS plots (FIG. 1G) and quantitation (FIG. 1H) of cells transfected with the indicated conditions showing fractions of unedited (gray), and cells edited by HDR (top right quadrant), NHEJ (bottom left quadrant) or HDR+NHEJ (bottom right quadrant). Error bars represent S.D. in FIG. 1H. FIG. 1I depicts results of modified-mRNA delivery of Rad52 and dn53BP1.

FIGS. 2A-2G demonstrate that Rad52 and dn53BP1 improve precise genomic modifications at various targeted loci. FIG. 2A depicts a schematic image of the approach utilized to detect DNA repair in bulked samples. A donor DNA containing a restriction site (PmeI) is used as a repair template in association with Cas9 and gRNAs targeting to different loci. After PCR amplification of the target regions, PCR products are digested with the restriction enzyme, giving rise to a lower band as a result of the cleaved PCR product. FIG. 2B, top panel depicts a gel image of cleaved PCR products generated after transfection of HEK 293 cells with Cas9, gRNA and donor template targeting JAK2, EMX1, HBB and CCR5 genes. FIG. 2B, bottom panel, depicts a bar graph showing percentage of the intensity of the lower bands in comparison to total DNA in HEK 293 cells for the different loci. FIG. 2C, top panel, depicts a gel of cleaved PCR products generated after transfection of human induced pluripotent stem (iPS) cells with Cas9, gRNA and donor template targeting the same genes previously described. FIG. 2C, bottom panel, depicts a bar graph showing percentage of the intensity of the lower bands in comparison to total DNA in iPS cells for the different loci. FIG. 2D depicts a bar graph showing percentage of repair calculated as previously described for two different cells lines of iPS cells derived from patients with a deletion (del37L) and a mutation (A353V) in the DKC1 gene. Correction of these mutations generates respectively a restriction site for XmnI and MspA1I, which were used to calculate the percentage of correction. FIG. 2E depicts sanger sequencing of corrected clones (SEQ ID NOS 39, 40, 41, 41, 41, 41, 41, 41, and 41, respectively, in order of appearance). Spacer sequence is highlighted in Magenta and PAM sequence is highlighted in Cyan. (Note the protospacer is in reverse orientation). DKC1^{A353V} mutation (C>T) is highlighted in Green. A silent mutation (C>T) is introduced in to the ssODN (highlighted in grey boxes) to destroy the PAM to abolish further cutting of repaired sequence by Cas9. FIG. 2F depicts Northern blot radiograph showing TERC and 18S (loading control) RNA levels in wild type (WT), DKC1^{A353V} and gene corrected iPS (DKC1#2AB3) cells. FIG. 2G depicts Southern blot telomere length analysis in WT, DKC1^{A353V} and gene corrected (DKC1#2AB3) iPS cell lines.

FIG. 3 depicts a schematic showing a reporter system to measure NHEJ (loss of B2M expression) and HDR (eGFP+). Guide targeting B2M and broken GFP were co-transfected together with Cas9 expression plasmids.

FIGS. 4A-4D demonstrate that HDR frequency can be optimized by multiple parameters. HEK293 cells with a broken GFP gene sequence were co-transfect with Cas9 [C] and gRNA [R] plasmids and a 100 bp single-stranded donor template [D] in order to obtain repaired GFP-positive cells. 20,000 cells were seeded in wells of a 96-well plate (day 1) and transfected 24 hours later (day 2). Cells were analyzed after 72 hours (day 4). FIG. 4A depicts a bar graph of percentage of GFP-positive cells after the first set of optimization using 5 concentrations of each plasmid: 25ng; 50ng; 100ng; 200ng; 500ng; and donor template concentration was fixed at 4 pmol. Same results were also represented as a heat map, where statistical significant conditions are marked by black line. FIG. 4B depicts representative FACs plots of each condition of the first set of optimization. FIG. 4C depicts graphs for the second set of optimizations—3 different numbers of cells were seeded (20,000; 10,000; 5,000) on 96-well plates, transfected (Cas9 and gRNA: 25ng) at day 1 and GFP expression was analyzed in 3 different days (day 3, d3; day 4, d4; and day 5, d5). Bar graph shows percentage of GFP-positive cells at d3, d4 and d5 after transfecting 20,000 cells (white bars), 10,000 cells (light grey bars) or 5,000 cells (dark grey bars). FIG. 4D depicts representative FACs plots of each condition of the second set of optimization.

FIGS. 5A-5C demonstrate that HDR frequency can be optimized by donor template orientation, concentration and length. FIG. 5A depicts a bar graph of percentage of GFP positive cells after transfection with either sense donor template [D] or antisense donor template [DR] independently in 2, 4 or 6 pmol and conditions where both were co-transfected (D 3 pmol+DR 3 pmol; D 4 pmol+DR 2 pmol; D 2 pmol+DR 4 pmol) or pre-annealed as a double-stranded DNA before transfecting (D 3 pmol+DR 3 pmol [iv anneal]). FIG. 5B depicts a bar graph of concentration curve for the sense 100 bp donor template. FIG. 5C, top: Donor templates with 100 bp [D100 bp], 142 bp [D142 bp] or 184 bp [D184 bp] of homology to the repaired gene were designed balanced (with same number of base pairs in each side [D142 bp (71/71); D184 bp (92/92)]) or imbalanced (different number of base pairs in each side [D142 bp (92/50); D184 bp (134/50)]). FIG. 5C, bottom: bar graph shows frequency of GFP expressing cells after transfection with all the donors. Significance was calculated using the One-way ANOVA: **p<0.01, ***p<0.001, ns, not significant. The bars represent mean values+s.e.m. Scale bars: 100 μm.

FIGS. 6A-6B demonstrate that re-screening of HDR inducers candidates, e.g., NHEJ inhibitors and HDR agonists, in HEK 293 cells and iPS cells. The same factors previously screened to increase HDR through the expression of GFP in a cell line with a broken GFP sequence were re-screened in HEK 293 cells and iPS cells by introducing a restriction site in the donor sequences. FIG. 6A depicts a bar graph of the percentage HDR in HEK 293 cells targeting both JAK2 and HBB. FIG. 6B depicts a bar graph of the percentage HDR in iPS cells targeting HBB. Percentage of repair was calculated based on the ratio of cleaved bands in comparison to total DNA. One-way ANOVA: *p<0.05, **p<0.01, ***p<0.001, ns, not significant. The bars represent mean values+s.e.m.

FIG. 7 depicts quantitation of HDR frequency post-optimization of all parameters tested in FIG. 4A-4D in multiple independent experiments (n=10).

FIGS. 8A-8D demonstrate that over-expression of RAD52 and dn53BP1 does not alter Cas9 specificity. FIG. 8A depicts off-target analysis by HTGTS for gRNA targeting CCR5 showing chromosomal location of on-target for gCCR5D (SEQ ID NO: 42) and gCCR5Q (SEQ ID NO: 43), and identified off-target sites (OT1-7) (SEQ ID NOS 44-50, respectively, in order of appearance) for gCCR5Q with mismatches shown in bold. Translocation junctions (of gCCR5D=52,719 gCCR5Q=79,677 junctions analyzed) and frequencies per 10,000 junctions are shown with data pooled from 3 independent experiments. FIGS. 8B-8D depict off-target analysis by HTGTS in presence of RAD52 and/or dn53BP1 showing translocation junction under indicated conditions (FIG. 8B), and microhomology distribution at the junctions with respect to RAG1 bait at the on-target sites for gCCR5D (FIG. 8C) and gCCR5Q (FIG. 8D) in presence of RAD52 and/or dn53BP1 In FIGS. 8C-8D the number of junctions with <11 bp microhomology (n) under each conditions are indicated. CR=Cas9+gRNA. Pooled data from 3 independent experiments normalized to 22,086 total junctions for each library is shown. Error bars represent S.E.M. Significance was calculated using 2-way ANOVA (*p<0.05, **p<0.01, ***p<0.0001). The top row of *'s corresponds to CR+RAD52, the middle row of *'s to CR+dn53, and the bottom row of *'s to CR+Rad52+dn53BP1.

FIGS. 9A-9C demonstrate that co-expression of RAD52 and dn53BP1 improves HDR frequency using Cas9-Nickase at multiple loci and in human cells. FIG. 9A depicts the quantification of HDR frequency following targeted repair of broken GFP with Cas9D10A nickase. Representative gel electrophoresis and quantification of HDR frequency at (FIG. 9B) EMX1 and (FIG. 9C) HBB locus in HEK293T cells. Experiments were performed in triplicates and pooled data from more than three independent experiments are shown. Error bars represent S.E.M. Significance was calculated using the One-way ANOVA (*p<0.05, **p<0.01, ***p<0.001). CR: Cas9+gRNA, CRD: CR+donor.

FIGS. 10A-10B demonstrate that co-expression of RAD52 and dn53BP1 improves multiplex-HDR in iPS cells. Simultaneous HDR-mediated gene editing at four loci using a multiplexed approach. Representative gel electrophoresis image (FIG. 10A) and quantification of HDR frequency (FIG. 10B) at four targeted loci in iPS cells. Experiments were performed in triplicates and pooled data from more than three independent experiments are shown. Error bars represent S.E.M. Significance was calculated using the Student's t-test (*p<0.05, **p<0.01, ***p<0.001). CR: Cas9+gRNA, CRD: CR+donor.

### DETAILED DESCRIPTION

As described herein, the inventors have found that the frequency of HDR-mediated gene modifications can be increased by using the combination of 1) NHEJ inhibitors and 2) HDR promoters/inducers. Furthermore, the inventors have identified specific compounds and classes of compounds that demonstrate surprising efficacy in accomplishing the necessary inhibition and/or promotion. Accordingly, in one aspect of any of the embodiments described herein is a method of altering a target sequence of a target nucleic acid molecule, the method comprising contacting the target nucleic acid molecule with: a) a nuclease; b) at least one inhibitor of non-homologous end joining (NHEJ); c) at least one agonist of homology-directed repair (HDR); and d) a template nucleic acid. In one aspect of any of the embodiments described herein is a method of altering a target sequence of a target nucleic acid molecule, the method comprising contacting the target nucleic acid molecule with: a) a nuclease; b) at least one inhibitor of non-homologous end joining (NHEJ); c) at least one agonist of homology-directed repair (HDR); and optionally, d) a template nucleic acid.

As used herein, “alteration of a target sequence” refers to the process of causing a directed change in a target sequence. The alteration can comprise any change in the sequence, e.g., an insertion, a deletion, an indel, a point mutation, a repair of a mutation, etc. In some embodiments of any of the aspects, the alteration of a target sequence can comprise insertion of, e.g., a wildtype sequence, a sequence endogenous to the species, and/or a sequence exogenous to the species. In some embodiments of any of the aspects, the alteration of a target sequence can comprise a repair of a mutation, e.g., a germline or acquired mutation, e.g., for therapeutic purposes. The target sequence is located in a target nucleic acid molecule. In some embodiments, the target nucleic acid molecule can be a chromosome. In some embodiments, the target nucleic acid molecule can be genomic DNA or mitochondrial DNA.

The alteration of the target sequence is accomplished by HDR, e.g., using the at least a portion of the template nucleic acid to repair a break in the target sequence caused by the nuclease. As used herein, “template nucleic acid” refers to a nucleic acid molecule comprising a sequence which is to be incorporated into the target nucleic acid molecule. The sequence to be incorporated can be introduced into the target nucleic acid molecule via homology directed repair at the target sequence, thereby causing an alteration of the target sequence, from the original target sequence to the sequence comprised by the template nucleic acid. Accordingly, the sequence comprised by the template nucleic acid can be, relative to the target sequence, an insertion, a deletion, an indel, a point mutation, a repair of a mutation, etc. The template nucleic acid can be, e.g., a single-stranded DNA molecule; a double-stranded DNA molecule; a DNA/RNA hybrid molecule; and a DNA/modRNA (modified RNA) hybrid molecule.

The template nucleic acid, in addition to the sequence which is to be incorporated into the target nucleic acid molecule, can comprise one or more regions flanking the sequence which is to be incorporated into the target nucleic acid molecule. The flanking regions can comprise sequences with homology to the target sequence and/or sequences flanking the target sequence, i.e., in order to hybridize with the target nucleic acid near the target sequence and permit HDR to occur. In some embodiments, the total size of the flanking region(s) is at least 100 bp. In some embodiments, the total size of the flanking region(s) is at least 150 bp. In some embodiments, the total size of the flanking region(s) is at least 184 bp. Design of template nucleic acids, particularly with respect to flanking region(s) is discussed further elsewhere herein and e.g., in Richardson et al., Nat. Biotech., 2016; which is incorporated by reference herein in its entirety.

Non-homologous end joining (NHEJ) is a process by which double-stranded breaks in DNA are repaired. Two ends generated by one or more DSBs are ligated together and since a template is not used, the repair typically generates changes in the sequence relative to the sequence that existed prior to the DSB's formation. As a process for DNA editing, NHEJ is not preferred due to the low likelihood of the introduced sequence being targeted as well as the high rate of mutation and low level of precision even when the template is inserted at the desired locus.

An inhibitor of NHEJ for use in the methods described herein can include an inhibitor of Ku70; an inhibitor of Ku80; an inhibitor of 53BP1; and/or an inhibitor of Lig4. In some embodiments of any of the aspects, an inhibitor of NHEJ for use in the methods described herein can include an inhibitor of Ku70; an inhibitor of Ku80; and/or an inhibitor of 53BP1.

“Ku70” (or “X-ray repair complementing defective repair in Chinese hamster cells 6” or “XRCC6”), in combination with “Ku80” (or “X-ray repair complementing defective repair in Chinese hamster cells 5” or “XRCC5”) are polypeptides that together, form the Ku heterodimer, which is part of the NHEJ process. The Ku heterodimer binds to DSB ends and is believed to form a scaffold for the other components of NHEJ at the DSB. Sequences for Ku70 are known for a number of species, e.g., human Ku70 (NCBI Gene ID: 2547), mRNA (e.g., NM_001288976.1) and polypeptide (e.g., NP_001275905). Sequences for Ku80 are known for a number of species, e.g., human Ku80 (NCBI Gene ID: 7520), mRNA (e.g., NM_021141.3) and polypeptide (e.g., NP_066964.1).

As used herein, “53BP1” or “p53-binding protein 1” refers to a protein in the NHEJ pathway that binds to DSB ends and is mutually antagonistic with BRCA1, thus exhibiting an inhibitory effect on HDR. Sequences for 53BP1 are known for a number of species, e.g., human 53BP1 (NCBI Gene ID: 7158), mRNA (e.g., NM_001141980.1) and polypeptide (e.g., NP_001135452.1 (SEQ ID NO: 12)).

As used herein, “Lig4” or “ligase IV” refers to a ligase that joins DSB ends during NHEJ. Sequences for Lig4 are known for a number of species, e.g., human Lig4 (NCBI Gene ID: 3981), mRNA (e.g., NM_001098268.1) and polypeptide (e.g., NP_001091738.1).

As used herein, the term “inhibitor” refers to an agent which can decrease the expression and/or activity of the targeted expression product, e.g. by at least 10% or more, e.g. by 10% or more, 50% or more, 70% or more, 80% or more, 90% or more, 95% or more, or 98% or more. The efficacy of an inhibitor of a particular target e.g. its ability to decrease the level and/or activity of the target can be determined, e.g. by measuring the level of an expression product and/or the activity of the target. Methods for measuring the level of a given mRNA and/or polypeptide are known to one of skill in the art, e.g. RT-PCR with primers can be used to determine the level of RNA and Western blotting with an antibody can be used to determine the level of a polypeptide. The activity of a NHEJ-promoting protein described herein can be determined by measuring the frequency of NHEJ, e.g. as described in the Examples herein. Changes in the amount and/or molecular weights of one or more targets, indicating cleavage of the target, are readily detected by western blot. In some embodiments, the inhibitor can be an inhibitory nucleic acid; an aptamer; an antibody reagent; an antibody; or a small molecule.

In some embodiments of any of the aspects, the inhibitor of Ku70; Ku80; 53BP1 and/or Lig4 can be an inhibitory nucleic acid. In some embodiments of any of the aspects, the inhibitor of Ku70 can be an inhibitory nucleic acid. In some embodiments of any of the aspects, the inhibitor of Ku80 can be an inhibitory nucleic acid. In some embodiments of any of the aspects, the inhibitor of 53BP1 can be an inhibitory nucleic acid. In some embodiments of any of the aspects, the inhibitor of Lig4 can be an inhibitory nucleic acid.

In some embodiments of any of the aspects, the inhibitor of Lig4 can be SCR7 (see, e.g., Chu et al. Nat Biotechnol. 2015 May; 33(5):543-8; and Srivastava, M. et al. Cell 151, 1474-1487 (2012); each of which is incorporated by reference herein in its entirety.

In some embodiments of any of the aspects, an inhibitor of 53BP1 can be a dominant-negative 53BP1 (dn53BP1) polypeptide. As used herein “dn53BP1” refers to a variant of 53BP1 which lacks the BRCT domain(s) but does comprise the Tudor domain(s) of wild-type 53BP1. In some embodiments of any of the aspects, the dn53BP1 can lack the residues corresponding to about residues 1774-1977 of SEQ ID NO:12. In some embodiments of any of the aspects, the dn53BP1 consists essentially of the sequence corresponding to about residues 1493-1537 of SEQ ID NO:12. In some embodiments of any of the aspects, the dn53BP1 consists essentially of the sequence corresponding to about residues 1218-1715 of SEQ ID NO:12. In some embodiments of any of the aspects, the dn53BP1 consists essentially of the sequence corresponding to about residues 1-1715 of SEQ ID NO:12. In some embodiments of any of the aspects, the dn53BP1 consists essentially of the sequence corresponding to about residues 1-1537 of SEQ ID NO:12. In some embodiments, a dn53BP1 polpeptide comprises only the Tudor domain(s) of wildtype 53BP1. Construction and design of dn53BP1 is discussed further at, e.g., Xie, A. et al. Mol Cell 28, 1045-1057 (2007); which is incorporated by reference here in its entirety. In some embodiments of any of the aspects, the target nucleic acid is contacted with the dn53BP1 polypeptide by delivering a dn53BP1 polypeptide to the target nucleic acid. In some embodiments of any of the aspects, the target nucleic acid is contacted with the dn53BP1 polypeptide by delivering a nucleic acid encoding the dn53BP1 polypeptide to the target nucleic acid.

Homology-directed repair is a process by which a DSB or nick in the DNA is repaired by hybridizing a template nucleic acid to the cut DNA and using a polymerase to construct the missing strand from the template sequence. HDR is preferred for DNA editing due to the extremely low error rate and the ability to change the target sequence with extreme precision.

An agonist of HDR for use in the methods described herein can include an agonist of BLM; an agonist of RAD52; and/or an agonist of RAD51.

As used herein, “Rad52” refers to a component of the HDR process that promotes assembly of Rad51 on ssDNA. Sequences for Rad52 are known for a number of species, e.g., human Rad52 (NCBI Gene ID: 5893), isoform a (mRNA (e.g., NM_001297419.1) and polypeptide (e.g., NP_001284348.1 (SEQ ID NO: 1)), isoform b (mRNA (e.g., NM_001297420.1) and polypeptide (e.g., NP_001284349.1 (SEQ ID NO: 2)), isoform c (mRNA (e.g., NM_001297421.1) and polypeptide (e.g., NP_001284350.1 (SEQ ID NO: 3)), and isoform d (mRNA (e.g., NM_001297422.1) and polypeptide (e.g., NP 001284351.1 (SEQ ID NO: 4)).

As used herein, “Rad51” refers to a RecA-like NTPase which is a component of the HDR process that promotes ATP-dependent DNA strand exchange. Sequences for Rad51 are known for a number of species, e.g., human Rad51 (NCBI Gene ID: 5888), mRNA (e.g., NM_133487.3) and polypeptide (e.g., NP_597994.3 (SEQ ID NO: 5), NP_002866.2 (SEQ ID NO: 6), NP_001157742.1 (SEQ ID NO: 7) and NP_001157741.1 (SEQ ID NO: 8).

As used herein, “Blm” or “Bloom syndrome RecQ like helicase” refers to a helicase which is a component of the HDR process that interacts with Rad51. Sequences for Blm are known for a number of species, e.g., human Blm (NCBI Gene ID: 641), mRNA (e.g., NM_000057.3) and polypeptide (e.g., NP_000048.1 (SEQ ID NO: 9). NP_001274176.1 (SEQ ID NO: 10), and NP_001274177.1 (SEQ ID NO: 11).

In some embodiments of any of the aspects described herein, the agonist of HDR can be an agonist of an agonist of RAD52 and/or an agonist of RAD51. In some embodiments of any of the aspects described herein, the agonist of HDR can be an agonist of an agonist of RAD52. In some embodiments of any of the aspects described herein, the agonist of HDR can be an agonist of RAD51.

As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, RAD52, e.g. its ability to increase the level and/or activity of RAD52 can be determined, e.g. by measuring the level of an expression product of RAD52 and/or the activity of RAD52 (or the rate of HDR as described in the Examples herein). Methods for measuring the level of a given mRNA and/or polypeptide are known to one of skill in the art, e.g. RTPCR with primers can be used to determine the level of RNA, and Western blotting with an antibody can be used to determine the level of a polypeptide.

Non-limiting examples of agonists of a given target, e.g., RAD52, can include RAD52 polypeptides or fragments thereof and nucleic acids encoding a RAD52 polypeptide or variants thereof. In some embodiments, the agonist of, e.g. RAD52 can be a RAD52 polypeptide. In some embodiments, the polypeptide agonist can be an engineered and/or recombinant polypeptide. In some embodiments, the polypeptide agonist can be a nucleic acid encoding polypeptide, e.g. a functional fragment thereof. In some embodiments of any of the aspects described herein, the nucleic acid can be comprised by a vector.

In some embodiments, the agonist of, e.g., Rad52, Rad51, and/or Blm can be a Rad52, Rad51, and/or Blm polypeptide, e.g., exogenous Rad52, Rad51, and/or Blm polypeptide. In some embodiments of any of the aspects, the target nucleic acid is contacted with exogenous Rad52, Rad51, and/or Blm polypeptide, e.g., Rad52, Rad51, and/or Blm polypeptide is produced in vitro and/or synthesized and purified Rad52, Rad51, and/or Blm polypeptide is provided to the target nucleic acid molecule. As used herein, “Rad52 polypeptide” can encompass any isoform of Rad52.

In some embodiments, the agonist of Rad52 can be a polypeptide comprising the sequence of any Rad52 isoform, e.g., SEQ ID NO: 1-4. In some embodiments, the agonist of Rad52 can be a polypeptide comprising the sequence of a Rad52 isoform, e.g., SEQ ID NO: 1-4 or a variant thereof. In some embodiments, the agonist of Rad52 can be a nucleic acid encoding a polypeptide comprising the sequence of Rad52 and/or a vector comprising a nucleic acid encoding a polypeptide comprising the sequence of Rad52. In some embodiments, the agonist of Rad52 can be a nucleic acid encoding a polypeptide comprising the sequence of Rad52 or a variant thereof and/or a vector comprising a nucleic acid encoding a polypeptide comprising the sequence of Rad52 or a variant thereof.

In some embodiments, the agonist of Rad51 can be a polypeptide comprising the sequence of Rad51, e.g., SEQ ID NO: 5-8. In some embodiments, the agonist of Rad51 can be a polypeptide comprising the sequence of Rad51, e.g., SEQ ID NO: 5-8 or a variant thereof. In some embodiments, the agonist of Rad51 can be a nucleic acid encoding a polypeptide comprising the sequence of Rad51 and/or a vector comprising a nucleic acid encoding a polypeptide comprising the sequence of Rad51. In some embodiments, the agonist of Rad51 can be a nucleic acid encoding a polypeptide comprising the sequence of Rad51 or a variant thereof and/or a vector comprising a nucleic acid encoding a polypeptide comprising the sequence of Rad51 or a variant thereof.

In some embodiments, the agonist of Blm can be a polypeptide comprising the sequence of Blm, e.g., SEQ ID NO: 9-11. In some embodiments, the agonist of Blm can be a polypeptide comprising the sequence of Blm, e.g., SEQ ID NO: 9-11 or a variant thereof. In some embodiments, the agonist of Blm can be a nucleic acid encoding a polypeptide comprising the sequence of Blm and/or a vector comprising a nucleic acid encoding a polypeptide comprising the sequence of Blm. In some embodiments, the agonist of Blm can be a nucleic acid encoding a polypeptide comprising the sequence of Blm or a variant thereof and/or a vector comprising a nucleic acid encoding a polypeptide comprising the sequence of Blm or a variant thereof.

In some embodiments of any of the aspects, ectopic polypeptide can be provided for use in the methods described herein by contacting the target nucleic acid with a nucleic acid encoding the ectopic polypeptide. A nucleic acid encoding a polypeptide can be, e.g., an RNA molecule, a plasmid, and/or an expression vector. In some embodiments of any of the aspects, the nucleic acid encoding a polypeptide can be an mRNA. In some embodiments of any of the aspects, the nucleic acid encoding a polypeptide can be a modified mRNA.

In some embodiments of any of the aspects, the Rad52, Rad51, and/or Blm polypeptide can be a constitutively active variant of the polypeptide. In some embodiments of any of the aspects, the agonist of Rad52 is ectopic Rad52 polypeptide or a constitutively active RAD52 polypeptide. In some embodiments of any of the aspects, the agonist of RAD51 is ectopic RAD51 polypeptide or a constitutively active RAD51 polypeptide. In some embodiments of any of the aspects, the agonist of RAD51 is constitutively active RAD51 polypeptide. In some embodiments of any of the aspects, the agonist of BLM is ectopic BLM polypeptide.

Rad51 and Rad52 are negatively regulated by phosphorylation at particular residues. Accordingly, constitutively active variants of, e.g. Rad51 and Rad52 can be provided by mutating one or more of those residues to prevent phosphorylation and thereof the ensuing negative regulation. Constitutively active variants can include, e.g., RAD51^{S309E}, RAD52^{Y104E}.

In some embodiments of any of the aspects, an agonist of HDR can be an adenovirus polypeptide or variant thereof. In some embodiments of any of the aspects, an agonist of HDR can be E1B55K and/or E4orf6. E1B55K and E4orf6 are adenovirus proteins that can increase the rate of HDR. See, e.g., Chu et al, Nat Biotechnol. 2015 May; 33(5):543-8; which is incorporated by reference herein in its entirety.

In some embodiments, the method and compositions described herein relate to a pairwise combination of agents as indicated in Table 1. In some embodiments, the method and compositions described herein relate to a pairwise combination of agents as indicated in Table 2.

TABLE 1
Possible pairwise combinations of agents indicated by “X”

| | | Agonist of HDR | | |
|---|---|---|---|---|
| | | Agonist of | Agonist of | Agonist of |
| | | Rad52 | Rad51 | Blm |
| Inhibitor | Inhibitor of Ku70 | X | X | X |
| of NHEJ | Inhibitor of Ku80 | X | X | X |
| | Inhibitor of 53BP1 | X | X | X |
| | Inhibitor of Lig4 | X | X | X |

TABLE 2
Certain embodiments of possible pairwise combinations
of agents indicated by “X”

| Agonist of HDR | | |
|---|---|---|
| Agonist of | Agonist of | Agonist of |
| Rad52 | Rad51 | Blm |

| | | |
|---|---|---|
| Inhibitor | Inhibitor of Ku70 | X |
| of NHEJ | Inhibitor of Ku80 | X |
| | Inhibitor of 53BP1 | X |
| | Inhibitor of Lig4 | |

In some embodiments of any of the aspects, the inhibitor of NHEJ is an inhibitor of 53BP1 and the agonist of HDR is an agonist of RAD52. In one aspect of any of the embodiments, described herein is a method of altering the sequence of a target nucleic acid molecule, the method comprising contacting the target nucleic acid molecule with: a) a nuclease; b) a template nucleic acid; and c) at least one inhibitor of 53BP1 and/or at least one agonist of RAD52. In one aspect of any of the embodiments, described herein is a method of altering the sequence of a target nucleic acid molecule, the method comprising contacting the target nucleic acid molecule with: a) a nuclease; b) a template nucleic acid; and c) at least one inhibitor of 53BP1 and at least one agonist of RAD52.

In one aspect of any of the embodiments, described herein is a method of altering the sequence of a target nucleic acid molecule, the method comprising contacting the target nucleic acid molecule with: a) a nuclease; and b) at least one agonist of RAD52. In some embodiments, the method can further comprise contacting the target nucleic acid molecule with an inhibitor of 53BP1.

As used herein, “nuclease” refers to an enzyme capable of cleaving the phosphodiester bonds between the nucleotide subunits of nucleic acids. Nucleases can be site-specific, i.e. site-specific nucleases cleave DNA bonds only after specifically binding to a particular sequence. Therefore, nucleases specific for a given target can be readily selected by one of skill in the art. Nucleases often cleave both strands of dsDNA molecule within several bases of each other, resulting in a double-stranded break (DSB). Exemplary nucleases include, but are not limited to Cas9; meganucleases; TALENs; zinc finger nucleases; Fokl cleavage domain; RNA-guided engineered nucleases; Cas9-derived nucleases; homing endonucleases (e.g. I-AniI, I-CreI aNd I-SCeI) and the like. Further discussion of the various types of nucleases and how their site-specificity can be engineered can be found, e.g. in Silva et al. Curr G ene Ther 2011 11:11-27; Gaj et al. Trends in Biotechnology 2013 31:397-405; Humbert et al. Critical Reviews in Biochemistry and Molecular Biology 2012 47:264-281; and Kim and Kim Nature 2014 doi: 10.1038/nrg3686, each of which is incorporated by reference herein in its entirety.

In some embodiments, the nuclease can be an engineered nuclease. As used herein, “engineered” refers to the aspect of having been manipulated by the hand of man. For example, a nuclease is considered to be “engineered” when the sequence of the nuclease is manipulated by the hand of man to differ from the sequence of the nuclease as it exists in nature. As is common practice and is understood by those in the art, progeny and copies of an engineered polynucleotide and/or polypeptide are typically still referred to as “engineered” even though the actual manipulation was performed on a prior entity.

In some embodiments of any of the aspects, the nuclease is a programmable nuclease. As used herein “programmable nuclease” refers to a nuclease that has been engineered to create a DSB or nick at a nucleic acid sequence that the native nuclease would not act upon, e.g. the sequence specificity of the nuclease has been altered. For example, Cas9-derived nucleases and nickases are targeted by means of guide nucleic acid molecules, which can be engineered to hybridize specifically to a desired target nucleic acid sequence (or a flanking sequence). By way of further non-limiting example, zinc finger nucleases can be targeted by a combinatorial assembly of multiple zinc finger domains with known DNA triplet specificities. Methods of engineering nucleases to achieve a desired sequence specificity are known in the art and are described, e.g., in Kim and Kim. Nature Reviews Genetics 2014 15:321-334; Kim et al. Genome Res. 2012 22:1327-1333; Belhaj et al. Plant Methods 2013 9:39; Umov et al. Nat Rev Genet 2010 11:636-646; Bogdanove et al. Science 2011 333:1843-6; Jinek et al. Science 2012 337:816-821; Silva et al. Curr Gene Ther 2011 11:11-27; Ran et al. Cell 2013 154:1380-9; Carlson et al. PNAS 212 109:17382-7, Guerts et al. Science 2009 325:433-3; Takasu et al. Insect Biochem Mol Biol 2010 40:759-765; and Watanabe et al. Nat. Commun. 2012 3; each of which is incorporated by reference herein in its entirety. By way of non-limiting example, the programmable nuclease can be Cas9; a Cas9 nickase mutant; TALEN; ZFNs; Cpf1; and/or SaCas9. In some embodiments of any of the aspects, the programmable nuclease is Cas9.

In some embodiments of any of the aspects, the nuclease can be an endonuclease. As used herein, “endonuclease” refers to an enzyme capable of denying the phosphodiester bonds between the nucleotide subunits of nucleic acids within a polynucleotide, e.g., cleaving a phosphodiester bond that is not either the 5′ or 3′ most bond present in the polynucleotide.

In some embodiments of any of the aspects, the nuclease can be a meganuclease. As used herein, “meganuclease” refers to endonucleases, which have a large recognition sequence (e.g., dsDNA sequences of 12-40 bp). Due to the size of the recognition sequences, meganucleases are particularly specific. Meganuclease specificity can be engineered. In some embodiments of any of the aspects, the meganuclease can be a LAGLIDADG (SEQ ID NO: 13) homing endonuclease.

In some embodiments, the nuclease can be specific for a portion of the target nucleic acid molecule at or near the target sequence, i.e., the nuclease can create a DSB or nick at a portion of the target nucleic acid molecule at or near the target sequence. In some embodiments, the nuclease can generate a DSB at the location where a portion of template nucleic acid is to be integrated in the target nucleic acid. In some embodiments, the nuclease can be specific for a portion of the target nucleic acid molecule located within a portion of the target nucleic acid sequence to which the template nucleic acid can specifically hybridize.

In some embodiments of any of the aspects, the method further comprises contacting the target nucleic acid molecule with a guide RNA that can hybridize to a portion of the target nucleic acid molecule. In some embodiments of any of the aspects, the nuclease is a Cas9 or Cas9-derived nuclease and the method further comprises contacting the target nucleic acid molecule with a guide RNA that can hybridize to a portion of the target nucleic acid molecule. Methods of designing and synthesizing guide RNAs (gRNAs) are known in the art and include, e.g., chemical alterations of the guide RNAs (see, e.g., Hendel et al. Nature Biotechnology 2015 33:985-989; Kiani et al. Nature Methods 2015 12:1051-1054; Smith et al. Genome Biol 2016 17:45; Doench et al. Nature Biotechnology 2016 34:184-191; each of which is incorporated herein by reference in its entirety).

When Cas9 nuclease (or Cas9-derived nuclease) is selected for use, the nuclease will generate a cut and/or nick where the guide RNA hybridizes to the target nucleic acid molecule. To promote HDR according to the methods described herein, the template nucleic acid can hybridize to the target nucleic acid molecule within 20 bp of where the guide RNA hybridizes to the target nucleic acid molecule. In some embodiments, the template nucleic acid can hybridize to the target nucleic acid molecule within 100 bp of where the guide RNA hybridizes to the target nucleic acid molecule. In some embodiments, the template nucleic acid can hybridize to the target nucleic acid molecule within 50 bp of where the guide RNA hybridizes to the target nucleic acid molecule. In some embodiments, the template nucleic acid can hybridize to the target nucleic acid molecule within 30 bp of where the guide RNA hybridizes to the target nucleic acid molecule. In some embodiments of any of the aspects, the portion of target nucleic acid molecule to which the template nucleic acid hybridizes can overlap with the portion of the target nucleic acid molecule to which the guide RNA hybridizes.

In some embodiments of any of the aspects, the contacting step occurs in a cell. In some embodiments of any of the aspects, the cell can be in vivo. In some embodiments of any of the aspects, the cell can be ex vivo. In some embodiments of any of the aspects, the cell can be isolated. By way of non-limiting example, the cell can be a eukaryotic cell; a mammalian cell; a human cell; a stem cell; an iPS cell; a hematopoietic cell; hematopoietic stem cell; hematopoietic progenitor cell or any combination of the foregoing. Agents can be introduced into a cell by any means known in the art, e.g., transfection, viral delivery, liposomal delivery, electroporation, cell squeeze, injection, endocytosis, and the like.

In some embodiments of any of the aspects, the target sequence is located at a locus, a coding gene sequence, a regulatory region, or a non-coding region.

In some embodiments of any of the aspects, the target sequence is comprised by the HBB gene. In some embodiments of any of the aspects, the target sequence is comprised by the ADA gene; IL-2Rγ gene; PNP gene; RAG-1 gene; RAG-2 gene; JAK2 gene; AK2 gene; DKC1 gene, or DCLRE1C gene. In some embodiments of any of the aspects, the alteration of the target sequence replaces and/or repairs a sequence associated with disease or disease susceptibility, e.g. the method described herein can be therapeutic.

In some embodiments of any of the aspects, the on-target or off-target cutting specificity of Cas9 activity is not altered by inclusion of the at least one inhibitor of NHEJ and/or at least one agonist of HDR. In some embodiments of any of the aspects, the on-target or off-target cutting specificity of Cas9 activity is altered by 10% or less (e.g., 10% or less, 9% or less, 8% or less, 7% or less, 6% or less, 5% or less, 4% or less, 3% or less, 2% or less, or 1% or less) by inclusion of the at least one inhibitor of NHEJ and/or at least one agonist of HDR.

Cells are more likely to undergo HDR, as opposed to NHEJ at certain points in the cell cycle. Accordingly, in some embodiments of any of the aspects, the methods described herein can further comprise contacting the cell with a cell cycle modulator. In some embodiments of any of the aspects, the cell cycle modulator increases the proportion of cells in late S or G2 phase. Such modulators are known in the art, e.g., aphidicolin and nocodazole. The relationship of the cell cycle to HDR, as well as exemplary modulators, are described, e.g., in Lin, S., et al Enhanced homology-directed human genome engineering by controlled timing of CRISPR/Cas9 delivery. eLife 4 (2014); which is incorporated by reference herein in it its entirety.

In some embodiments of any of the aspects, the methods described herein can further comprise contacting the cell with at least one factor that increases the survival, maintenance, and/or expansion of hematopoietic stem and progenitor cells. Such factors can include, by way of non-limiting example, the compounds and combinations of compounds described in International Patent Application No. PCT/US2016/039303.

In some embodiments of any of the aspects, the frequency of HDR is increased by the methods described herein at least 1.25 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 1.5 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 1.75 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 2 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 5 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 10 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 20 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 30 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 50 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 80 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 90 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 99 fold relative to the frequency of HDR in the absence of the at least one inhibitor of non-homologous end joining (NHEJ) and the at least one agonist of homology-directed repair (HDR). In some embodiments of any of the aspects, the frequency of HDR is increased by the methods described herein at least 100 fold relative to the frequency of HDR in the absence of the at least one inhibitor of non-homologous end joining (NHEJ) and the at least one agonist of homology-directed repair (HDR).

In one aspect of any of the embodiments, described herein is a composition comprising: a) at least one inhibitor of non-homologous end joining (NHEJ); and/or b) at least one agonist of homology-directed repair (HDR). In one aspect of any of the embodiments, described herein is a composition comprising: a) at least one inhibitor of non-homologous end joining (NHEJ); and b) at least one agonist of homology-directed repair (HDR).

In one aspect of any of the embodiments, described herein is a kit comprising: a) a cell comprising a target nucleic acid molecule and/or a nuclease; b) at least one inhibitor of non-homologous end joining (NHEJ); and/or c) at least one agonist of homology-directed repair (HDR). In one aspect of any of the embodiments, described herein is a kit comprising: a) a cell comprising a target nucleic acid molecule and/or a nuclease; b) at least one inhibitor of non-homologous end joining (NHEJ); and c) at least one agonist of homology-directed repair (HDR). In some embodiments of any of the aspects, the inhibitor and/or agonist are expressed from a nucleic acid molecule comprised by the cell.

A kit is any manufacture (e.g., a package or container) comprising at least one reagent, e.g., an inhibitor of NHEJ or agonist of HDR, the manufacture being promoted, distributed, or sold as a unit for performing the methods described herein. The kits described herein can optionally comprise additional components useful for performing the methods described herein. By way of example, the kit can comprise fluids and compositions (e.g., buffers, dNTPs, etc.) suitable for performing one or more of the reactions according to the methods described herein, an instructional material which describes performance of a method as described herein, and the like. Additionally, the kit may comprise an instruction leaflet and/or may provide information as to the relevance of the obtained results.

Further described herein is the discovery that the ratio and/or relative concentration of the elements required for HDR can exert a significant influence on the efficiency and/or frequency of HDR, e.g., as compared to the efficiency and/or frequency of NHEJ. Provided herein are methods for of altering a target sequence of a target nucleic acid molecule relating to ratios and/or relative concentrations that display surprising results. These methods can be combined, without limitation with the foregoing methods relating to inhibitors of NHEJ and/or agonists of HDR.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the ratio of the Cas9 nuclease:gRNA is about 1:4 or greater. In some embodiments of any of the aspects, the ratio of the Cas9 nuclease:gRNA is 1:4 or greater. In some embodiments of any of the aspects, the ratio of the Cas9 nuclease:gRNA is from about 1:4 to about 8:1. In some embodiments of any of the aspects, the ratio of the Cas9 nuclease:gRNA is from 1:4 to 8:1.

In some embodiments of any of the aspects, the concentration of the Cas9 nuclease does not exceed about 200 ng/5000 cells. In some embodiments of any of the aspects, the concentration of the Cas9 nuclease does not exceed 200 ng/5000 cells. In some embodiments of any of the aspects, the concentration of the gRNA does not exceed about 100 ng/5000 cells. In some embodiments of any of the aspects, the concentration of the gRNA does not exceed 100 ng/5000 cells. In some embodiments of any of the aspects, the concentration of the Cas9 nuclease does not exceed about 200 ng/5000 cells and the concentration of the gRNA does not exceed about 100 ng/5000 cells. In some embodiments of any of the aspects, the concentration of the Cas9 nuclease does not exceed 200 ng/5000 cells and the concentration of the gRNA does not exceed 100 ng/5000 cells.

In some embodiments of any of the aspects, the concentration of the template nucleic acid is about 2 pmol/5000 cells or greater. In some embodiments of any of the aspects, the concentration of the template nucleic acid is about 2 pmol/5000 cells or greater. In some embodiments of any of the aspects, the concentration of the template nucleic acid is about 20 pmol/5000 cells or less. In some embodiments of any of the aspects, the concentration of the template nucleic acid is 20 pmol/5000 cells or less.

In some embodiments of any of the aspects, the concentration of the template nucleic acid is from about 2 pmol/5000 cells to about 20 pmol/5000 cells. In some embodiments of any of the aspects, the concentration of the template nucleic acid is from 2 pmol/5000 cells to 20 pmol/5000 cells. In some embodiments of any of the aspects, the concentration of the template nucleic acid is from about pmol/5000 cells to about 12 pmol/5000 cells. In some embodiments of any of the aspects, the concentration of the template nucleic acid is from 2 pmol/5000 cells to 12 pmol/5000 cells. In some embodiments of any of the aspects, the concentration of the template nucleic acid is from about 4 pmol/5000 cells to about 20 pmol/5000 cells. In some embodiments of any of the aspects, the concentration of the template nucleic acid is from 4 pmol/5000 cells to 20 pmol/5000 cells. In some embodiments of any of the aspects, the concentration of the template nucleic acid is from about 4 pmol/5000 cells to about 12 pmol/5000 cells. In some embodiments of any of the aspects, the concentration of the template nucleic acid is from 4 pmol/5000 cells to 12 pmol/5000 cells.

In some embodiments of any of the aspects, the template nucleic acid has a portion with homology to the target nucleic acid molecule that is greater than about 100 bp in length. In some embodiments of any of the aspects, the template nucleic acid has a portion with homology to the target nucleic acid molecule that is about 142 bp or greater in length. In some embodiments of any of the aspects, the template nucleic acid has a portion with homology to the target nucleic acid molecule that is about 184 bp or greater in length. In some embodiments of any of the aspects, the template nucleic acid has a portion with homology to the target nucleic acid molecule that is greater than 100 bp in length. In some embodiments of any of the aspects, the template nucleic acid has a portion with homology to the target nucleic acid molecule that is 142 bp or greater in length. In some embodiments of any of the aspects, the template nucleic acid has a portion with homology to the target nucleic acid molecule that is 184 bp or greater in length.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the concentration of the template nucleic acid is about 2 pmol/5000 cells or greater. In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the concentration of the template nucleic acid is 2 pmol/5000 cells or greater. In some embodiments of any of the aspects, the concentration of the template nucleic acid is from about 2 pmol/5000 cells to about 20 pmol/5000 cells. In some embodiments of any of the aspects, the concentration of the template nucleic acid is from 2 pmol/5000 cells to 20 pmol/5000 cells. In some embodiments of any of the aspects, the concentration of the template nucleic acid is from about pmol/5000 cells to about 12 pmol/5000 cells. In some embodiments of any of the aspects, the concentration of the template nucleic acid is from 2 pmol/5000 cells to 12 pmol/5000 cells. In some embodiments of any of the aspects, the concentration of the template nucleic acid is from about 4 pmol/5000 cells to about 20 pmol/5000 cells. In some embodiments of any of the aspects, the concentration of the template nucleic acid is from 4 pmol/5000 cells to 20 pmol/5000 cells. In some embodiments of any of the aspects, the concentration of the template nucleic acid is from about 4 pmol/5000 cells to about 12 pmol/5000 cells. In some embodiments of any of the aspects, the concentration of the template nucleic acid is from 4 pmol/5000 cells to 12 pmol/5000 cells.

In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the template nucleic acid has a portion with homology to the target nucleic acid molecule that is greater than about 100 bp in length. In one aspect of any of the embodiments, described herein is a method of altering a target sequence of a target nucleic acid molecule, the method comprising contacting the target nucleic acid molecule with: a) a Cas9 nuclease; b) a guide RNA (gRNA) that can hybridize to a portion of the target nucleic acid molecule; and c) a template nucleic acid; wherein the template nucleic acid has a portion with homology to the target nucleic acid molecule that is greater than 100 bp in length. In some embodiments of any of the aspects, the template nucleic acid has a portion with homology to the target nucleic acid molecule that is about 142 bp or greater in length. In some embodiments of any of the aspects, the template nucleic acid has a portion with homology to the target nucleic acid molecule that is about 184 bp or greater in length. In some embodiments of any of the aspects, the template nucleic acid has a portion with homology to the target nucleic acid molecule that is 142 bp or greater in length. In some embodiments of any of the aspects, the template nucleic acid has a portion with homology to the target nucleic acid molecule that is 184 bp or greater in length.

In some embodiments of any of the aspects, the template nucleic acid has a portion with homology to the sense strand of the target nucleic acid molecule, e.g., it hybridizes to the antisense strand.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience; certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models. A subject can be male or female.

As used herein, “exposing” refers to directing or pointing an agent at a cell and/or contacting a cell with the agent. For example, exposing a cell to a source of radiation can comprise directing radiation towards the cell while exposing a cell to a proteinaceous agent can comprise contacting the cell with the agent. As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art.

As used herein, the term “hybridization” refers to the formation of one or more complementary base pairs between two nucleic acids, e.g., two complementary or substantially complementary nucleic acids strands annealing by base pair interactions. In some embodiments, conditions for hybridization (e.g., between a template and a target) may vary based of the length and sequence of a template or the portion thereof that is complementary or substantially complementary to the target. In some embodiments, conditions for hybridization are based upon a Tₘ (e.g., a calculated Tₘ.) of a template. In some embodiments, the methods described herein can be conducted at a temperature which is lower than the Tₘ (e.g., a calculated Tₘ) for a template. In some embodiments, a Tₘ can be determined using any of a number of algorithms (e.g., OLIGO™ (Molecular Biology Insights Inc. Colorado) primer design software and VENTRO NTI™ (Invitrogen, Inc. California) design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html). In some embodiments, the Tₘ of a template can be calculated using following formula, which is used by NetPrimer software and is described in more detail in Frieir et al. PNAS 1986 83:9373-9377 which is incorporated by reference herein in its entirety.

Tₘ=ΔH/(ΔS+R*ln(C/4))+16.6 log([K⁺]/(1+0.7[K⁺]))−273.15

wherein, ΔH is enthalpy for helix formation; ΔS is entropy for helix formation; R is molar gas constant (1.987 cal/° C.*mol); C is the nucleic acid concentration; and [K⁻] is salt concentration. In some embodiments, the closer a hybridizaton temperature is to the Tₘ, the more specific is the hybridization.

As used herein, “specific” when used in the context of the hybridization of a template nucleic acid sequence specific for a target sequence refers to a level of complementarity between the template and the target such that there exists an annealing temperature at which the template will anneal to the target sequence (or flanking sequence) and will not anneal to non-target sequences present in a sample.

As used herein, the term “complementary” refers to the ability of nucleotides to form hydrogen-bonded base pairs. In some embodiment, complementary refers to hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. As used herein, “substantially complementary” refers to a nucleic acid molecule or portion thereof (e.g. a template) having at least 90% complementarity over the entire length of the molecule or portion thereof with a second nucleotide sequence, e.g. 90% complementary, 95% complementary, 98% complementary, 99% complementary, or 100% complementary. As used herein, “substantially identical” refers to a nucleic acid molecule or portion thereof having at least 90% identity over the entire length of a the molecule or portion thereof with a second nucleotide sequence, e.g. 90% identity, 95% identity, 98% identity, 99% identity, or 100% identity.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the specific polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide, which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are also known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

In some embodiments, a nucleic acid encoding a polypeptide as described herein (e.g. a Rad52 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence, which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The term “exogenous” refers to a substance present in a cell other than its native source. The term “exogenous” when used herein can refer to a nucleic acid (e.g. a nucleic acid encoding a payload polypeptide) or a polypeptide (e.g., a payload polypeptide) that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in low amounts and one wishes to increase the amount of the nucleic acid or polypeptide in the cell or organism. In contrast, the term “endogenous” refers to a substance that is native to the biological system or cell (e.g. the microbial cell and/or target cell). As used herein. “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. In some embodiments, an antibody reagent can be a single domain antibody. In some embodiments of any of the aspects, the antibody reagent can be a single chain antibody reagent, e.g., one which, as a single polypeptide chain, can specifically bind the target antigen (e.g. nanobodies, VNA, and VHH).

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody, e.g., single domain antibody (VHH) can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

Inhibitors of the expression of a given gene can be an inhibitory nucleic acid. In some embodiments, the inhibitory nucleic acid is an inhibitory RNA (iRNA). As used herein, the term “iRNA” refers to any type of interfering RNA, including but are not limited to RNAi, siRNA, shRNA, endogenous microRNA and artificial microRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of the expression and/or activity of a target gene described herein. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNAs, e.g., modified mRNAs suitable for use in the methods described herein (e.g., for use in gRNAs and/or template nucleic acid molecules) are known in the art and can include, by way of non-limiting example, N6-Methyladenosine-5′-Triphosphate; 5-Methylcytidine-5′-Triphosphate; 2′-O-Methyladenosine-5′-Triphosphate; 2′-O-Methylcytidine-5′-Triphosphate; 2′-O-Methylguanosine-5′-Triphosphate; 2′-O-Methyluridine-5′-Triphosphate; Pseudouridine-5′-Triphosphate; Inosine-5′-Triphosphate; 2′-O-Methylinosine-5′-Triphosphate; 5-Methyluridine-5′-Triphosphate; 4-Thiouridine-5′-Triphosphate; 2-Thiouridine-5′-Triphosphate; 5,6-Dihydrouridine-5′-Triphosphate; 2-Thiocytidine-5′-Triphosphate; N1-Methylguanosine-5′-Triphosphate; 2′-O-Methylpseudouridine-5′-Triphosphate; N1-Methyladenosine-5′-Triphosphate; 2′-O-Methyl-5-methyluridine-5′-Triphosphate; N4-Methylcytidine-5′-Triphosphate; N1-Methylpseudouridine-5′-Triphosphate; 5,6-Dihydro-5-Methyluridine-5′-Triphosphate; 5-Formylcytidine-5′-Triphosphate; 5-Hydroxymethylcytidine-5′-Triphosphate; 5-Hydroxycytidine-5′-Triphosphate; 5-Hydroxyuridine-5′-Triphosphate; 5-Methoxyuridine-5′-Triphosphate; and 5-Carboxymethylesteruridine-5′-Triphosphate. Modified mRNAs and methods of making them are described, e.g., in International Patent Publications WO2012/135805; WO2012/019168; WO2013/151666; WO2013/151736; WO2013/151672; WO2013/151668; WO2013/151670; WO2013/151665; WO2013/096709; WO2013/039861; WO2013/090186; WO2014/093924; WO2015/051173; WO2015/089511; and WO2015/006747; U.S. Pat. Nos. 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,754,062; 8,680,069; and 8,664,194; each of which is incorporated by reference herein in its entirety.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. condition. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route, which results in at least partial delivery of the agent at a desired site. Pharmaceutical compositions comprising the compounds disclosed herein can be administered by any appropriate route, which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier. 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

Some embodiments of the technology described herein can be defined according to any of the following numbered paragraphs:
1. A method of altering a target sequence of a target nucleic acid molecule, the method comprising contacting the target nucleic acid molecule with:
   a. a nuclease;
   b. at least one inhibitor of non-homologous end joining (NHEJ);
   c. at least one agonist of homology-directed repair (HDR); and
   d. a template nucleic acid.
2. The method of paragraph 1, wherein the inhibitor of NHEJ is selected from the group consisting of:
   an inhibitor of Ku70, an inhibitor of Ku80, and an inhibitor of 53BP1.
3. The method of any of paragraphs 1-2, wherein the agonist of HDR is selected from the group consisting of:
   an agonist of RAD52 and an agonist of RAD51.
4. The method of any of paragraphs 1-2, wherein the agonist of HDR is selected from the group consisting of:
   an agonist of RAD52; an agonist of RAD51; and an agonist of BLM.
5. The method of any of paragraphs 1-4, wherein the inhibitor of NHEJ is an inhibitor of 53BP1 and the agonist of HDR is an agonist of Rad52.
6. A method of altering the sequence of a target nucleic acid molecule, the method comprising contacting the target nucleic acid molecule with:
   a. a nuclease;
   b. a template nucleic acid; and
   c. at least one inhibitor of 53BP1 and/or at least one agonist of RAD52.
7. The method of paragraph 6, wherein the target nucleic acid molecule is contacted with at least one inhibitor of 53BP1 and at least one agonist of RAD52.
8. A method of altering the sequence of a target nucleic acid molecule, the method comprising contacting the target nucleic acid molecule with:
   a. a nuclease; and
   b. at least one agonist of RAD52.
9. The method of paragraph 8, further comprising contacting the target nucleic acid molecule with an inhibitor of 53BP1.
10. The method of any of paragraphs 1-9, wherein the agonist of Rad52 is ectopic Rad52 polypeptide or a constitutively active RAD52 polypeptide.
11. The method of any of paragraphs 1-10, wherein the agonist of RAD51 is ectopic RAD51 polypeptide or a constitutively active RAD51 polypeptide.
12. The method of any of paragraphs 1-10, wherein the agonist of RAD51 is constitutively active RAD51 polypeptide.
13. The method of any of paragraphs 1-12, wherein the agonist of BLM is ectopic BLM polypeptide.
14. The method of any of paragraphs 10-13, wherein the target nucleic acid is contacted with the ectopic polypeptide by delivering a polypeptide to the target nucleic acid.
15. The method of any of paragraphs 10-13, wherein the target nucleic acid is contacted with the ectopic polypeptide by delivering a nucleic acid encoding the polypeptide to the target nucleic acid.
16. The method of any of paragraphs 1-15, wherein the inhibitor of NHEJ is an inhibitor of Lig4.
17. The method of paragraph 16, wherein the inhibitor of Lig4 is SCR7.
18. The method of any of paragraphs 1-17, wherein the target nucleic acid molecule is contacted with at least one agonist of HDR selected from E1B55K and E4orf6.
19. The method of any of paragraphs 1-18, wherein the inhibitor of Ku70 is an inhibitory nucleic acid.
20. The method of any of paragraphs 1-19, wherein the inhibitor of Ku80 is an inhibitory nucleic acid.
21. The method of any of paragraphs 1-20, wherein the inhibitor of 53BP1 is an inhibitory nucleic acid or a dominant-negative 53BP1 (dn53BP1) polypeptide.
22. The method of paragraph 21, wherein the target nucleic acid is contacted with the dn53BP1 polypeptide by delivering a polypeptide to the target nucleic acid.
23. The method of paragraph 21, wherein the target nucleic acid is contacted with the dn53BP1 polypeptide by delivering a nucleic acid encoding the polypeptide to the target nucleic acid.
24. The method of any of paragraphs 1-23, wherein the nucleic acid encoding a polypeptide is an mRNA.
25. The method of paragraph 24, wherein the mRNA is a modified mRNA.
26. The method of any of paragraphs 1-25, wherein the nuclease is a programmable nuclease.
27. The method of paragraph 26, wherein the programmable nuclease is selected from the group consisting of
   Cas9; a Cas9 nickase mutant; TALEN; ZFNs; Cpf1; and SaCas9.
28. The method of paragraph 26, wherein the programmable nuclease is Cas9.
29. The method of any of paragraphs 26-28, wherein the method further comprises contacting the target nucleic acid molecule with a guide RNA that can hybridize to a portion of the target nucleic acid molecule.
30. The method of any of paragraphs 26-28, wherein the nuclease is a Cas9 or Cas9-derived nuclease and the method further comprises contacting the target nucleic acid molecule with a guide RNA that can hybridize to a portion of the target nucleic acid molecule.
31. The method of any of paragraphs 1-25, wherein the nuclease is a meganuclease.
32. The method of any of paragraphs 1-31, wherein the template nucleic acid is selected from the group consisting of:
   a single-stranded DNA molecule; a double-stranded DNA molecule; a DNA/RNA hybrid molecule; and a DNA/modRNA hybrid molecule.
33. The method of any of paragraphs 1-32, wherein the contacting step occurs in a cell.
34. The method of paragraph 33, wherein the cell is a eukaryotic cell.
35. The method of paragraph 34, wherein the cell is a mammalian cell.
36. The method of paragraph 35, wherein the cell is a human cell.
37. The method of any of paragraphs 33-36, wherein the cell is a stem cell or iPSC.
38. The method of any of paragraphs 33-37, wherein the cell is a hematopoietic cell, hematopoietic stem cell, or hematopoietic progenitor cell.
39. The method of any of paragraphs 1-38, wherein the target nucleic acid molecule is a chromosome.
40. The method of any of paragraphs 1-39, wherein the target sequence is located in the genomic DNA or the mitochondrial DNA.
41. The method of any of paragraphs 1-40, wherein the target sequence is located at a locus, a coding gene sequence, or a regulatory region.
42. The method of any of paragraphs 1-41, wherein the target sequence is comprised by the HBB gene.
43. The method of any of paragraphs 1-41, wherein the target sequence is comprised by the ADA gene; IL-2Rγ gene; PNP gene; RAG-1 gene; RAG-2 gene; JAK3 gene; AK2 gene; or DCLRE1C gene.
44. The method of any of paragraphs 1-43, wherein the on-target or off-target cutting specificity of Cas9 activity is not altered by inclusion of the at least one inhibitor of NHEJ and/or at least one agonist of HDR.
45. The method of any of paragraphs 34-44, further comprising contacting the cell with a cell cycle modulator.
46. The method of paragraph 45, wherein the cell cycle modulator increases the proportion of cells in late S or G2 phase.
47. The method of any of paragraphs 34-46, further comprising contacting the cell with at least one factor that increases the survival, maintenance, and/or expansion of hematopoietic stem and progenitor cells.
48. The method of any of paragraphs 1-47, wherein the frequency of HDR is increased at least 1.25 fold relative to the frequency of HDR in the absence of the at least one inhibitor of non-homologous end joining (NHEJ) and the at least one agonist of homology-directed repair (HDR).
49. A composition comprising:
   a) at least one inhibitor of non-homologous end joining (NHEJ); and/or
   b) at least one agonist of homology-directed repair (HDR).
50. A kit comprising:
   a) a cell comprising a target nucleic acid molecule and/or a nuclease;
   b) at least one inhibitor of non-homologous end joining (NHEJ); and/or
   c) at least one agonist of homology-directed repair (HDR).
51. The kit of paragraph 50, wherein the inhibitor and/or agonist are expressed from a nucleic acid molecule comprised by the cell.
52. The kit or composition of any of paragraphs 49-51, wherein the inhibitor of NHEJ is selected from the group consisting of:
   an inhibitor of Ku70; an inhibitor of Ku80; and an inhibitor of 53BP1.
53. The kit or composition of any of paragraphs 49-52, wherein the agonist of HDR is selected from the group consisting of:
   an agonist of RAD52 and an agonist of RAD51.
54. The kit or composition of any of paragraphs 49-53, wherein the agonist of HDR is selected from the group consisting of:
   an agonist of RAD52; an agonist of RAD51; and an agonist of BLM.
55. The kit or composition of any of paragraphs 49-54, wherein the inhibitor of NHEJ is an inhibitor of 53BP1 and the agonist of HDR is an agonist of Rad52.
56. The kit or composition of any of paragraphs 49-55, wherein the agonist of RAD52 is ectopic Rad52 polypeptide or a constitutively active RAD52 polypeptide.
57. The kit or composition of any of paragraphs 49-56, wherein the agonist of RAD51 is ectopic RAD51 polypeptide or a constitutively active RAD51 polypeptide.
58. The kit or composition of any of paragraphs 49-56, wherein the agonist of RAD51 is constitutively active RAD51 polypeptide.
59. The kit or composition of any of paragraphs 49-58, wherein the agonist of BLM is ectopic BLM polypeptide.
60. The kit or composition of any of paragraphs 49-59, further comprising a nucleic acid encoding the ectopic polypeptide.
61. The kit or composition of any of paragraphs 49-60, wherein the inhibitor of NHEJ is an inhibitor of Lig4.
62. The kit or composition of paragraph 62, wherein the inhibitor of Lig4 is SCR7.
63. The kit or composition of any of paragraphs 49-62, further comprising at least one agonist of HDR selected from E1B55K and E4orf6.
64. The kit or composition of any of paragraphs 49-63, wherein the inhibitor of Ku70 is an inhibitory nucleic acid.
65. The kit or composition of any of paragraphs 49-64, wherein the inhibitor of Ku80 is an inhibitory nucleic acid.
66. The kit or composition of any of paragraphs 49-65, wherein the inhibitor of 53BP1 is an inhibitory nucleic acid or a dominant-negative 53BP1 (dn53BP1) polypeptide.
67. The kit or composition of any of paragraphs 49-66, further comprising a nucleic acid encoding the dn53BP1 polypeptide.
68. The kit or composition of any of paragraphs 49-67, wherein the nucleic acid encoding a polypeptide is an mRNA.
69. The kit or composition of any of paragraphs 49-68, wherein the mRNA is a modified mRNA.
70. A method of altering a target sequence of a target nucleic acid molecule, the method comprising contacting the target nucleic acid molecule with:
   a. a Cas9 nuclease;
   b. a guide RNA (gRNA) that can hybridize to a portion of the target nucleic acid molecule; and
   c. a template nucleic acid;
   wherein the ratio of the Cas9 nuclease:gRNA is 1:4 or greater.
71. The method of paragraph 70 wherein the ratio of the Cas9 nuclease:gRNA is 1:4 to 8:1.
72. The method of any of paragraphs 70-71, wherein the concentration of the Cas9 nuclease does not exceed 200 ng/5000 cells.
73. The method of any of paragraphs 70-72, wherein the concentration of the gRNA does not exceed 100 ng/5000 cells.
74. The method of any of paragraphs 70-73, wherein the concentration of the Cas9 nuclease does not exceed 200 ng/5000 cells and the concentration of the gRNA does not exceed 100 ng/5000 cells.
75. The method of any of paragraphs 70-74, wherein the concentration of the template nucleic acid is 2 pmol/5000 cells or greater.
76. The method of any of paragraphs 70-75, wherein the concentration of the template nucleic acid is 20 pmol/5000 or less.
77. The method of any of paragraphs 70-76, wherein the concentration of the template nucleic acid is from 2 pmol/5000 cells to 20 pmol/5000 cells.
78. The method of any of paragraphs 70-77, wherein the concentration of the template nucleic acid is from 2 pmol/5000 cells to 12 pmol/5000 cells.
79. The method of any of paragraphs 70-78, wherein the concentration of the template nucleic acid is from 4 pmol/5000 cells to 20 pmol/5000 cells.
80. The method of any of paragraphs 70-79, wherein the concentration of the template nucleic acid is from 4 pmol/5000 cells to 12 pmol/5000 cells.
81. The method of any of paragraphs 70-80, wherein the template nucleic acid has a portion with homology to the target nucleic acid molecule that is greater than 100 bp in length.
82. The method of any of paragraphs 70-81, wherein the template nucleic acid has a portion with homology to the target nucleic acid molecule that is 142 bp or greater in length.
83. The method of any of paragraphs 70-82, wherein the template nucleic acid has a portion with homology to the target nucleic acid molecule that is 184 bp or greater in length.
84. A method of altering a target sequence of a target nucleic acid molecule, the method comprising contacting the target nucleic acid molecule with:
   a. a Cas9 nuclease;
   b. a guide RNA (gRNA) that can hybridize to a portion of the target nucleic acid molecule; and
   c. a template nucleic acid;
   wherein the concentration of the template nucleic acid is 2 pmol/5000 cells or greater.
85. The method of paragraph 84, wherein the concentration of the template nucleic acid is from 2 pmol/5000 cells to 20 pmol/5000 cells.
86. The method of any of paragraphs 84-85, wherein the concentration of the template nucleic acid is from 2 pmol/5000 cells to 12 pmol/5000 cells.
87. The method of any of paragraphs 84-86, wherein the concentration of the template nucleic acid is from 4 pmol/5000 cells to 20 pmol/5000 cells.
88. The method of any of paragraphs 84-87, wherein the concentration of the template nucleic acid is from 4 pmol/5000 cells to 12 pmol/5000 cells.
89. A method of altering a target sequence of a target nucleic acid molecule, the method comprising contacting the target nucleic acid molecule with:
   a. a Cas9 nuclease;
   b. a guide RNA (gRNA) that can hybridize to a portion of the target nucleic acid molecule; and
   c. a template nucleic acid;
   wherein the template nucleic acid has a portion with homology to the target nucleic acid molecule that is greater than 100 bp in length.
90. The method of paragraph 89, wherein the template nucleic acid has a portion with homology to the target nucleic acid molecule that is 142 bp or greater in length.
91. The method of any of paragraphs 89-90, wherein the template nucleic acid has a portion with homology to the target nucleic acid molecule that is 184 bp or greater in length.
92. The method of any of paragraphs 70-91, wherein the template nucleic acid has a portion with homology to the sense strand of the target nucleic acid molecule.

### EXAMPLES

### Example 1: Transient Manipulation of DNA Damage Repair Choice Improves CRISPR/Cas9-Meduated Homology-Directed Repair

The CRISPR/Cas9 system allows efficient gene ablation through error-prone non-homologous end joining DNA repair. Very low efficiency of homology-directed DNA repair (HDR), however, is the bottleneck in correcting genetic mutations of clinical relevance. Described herein is that transient ectopic expression of Rad52 and/or dominant negative form of 53BP1 (dn53BP1) achieves HDR-mediated gene editing with 20-40% efficacy at multiple loci in human cells (including patient-specific iPS cells). Off-target analyses demonstrate that expression of Rad52 and dn53BP1 does not alter Cas9 specificity or off-target activity.

Repurposing type II bacterial CRISPR system as a genome-editing tool¹ has provided a robust technology for site-directed genome editing in mammalian cells²⁻⁴ including at disease relevant loci in primary cells⁵⁻¹². Mammalian cells repair DNA double strand breaks (DSB) by multiple pathways including the error prone non-homologous end-joining (NHEJ) pathway. Efficient DSB generated by Cas9 at the target site, followed by repair through NHEJ pathway, allows for robust gene ablation caused by frameshift mutations resulting from InDels. Alternatively, in the presence of a homologous DNA template, precise gene editing can be achieved through the homology-directed repair (HDR) pathway. Utilization of this pathway combined with repair templates containing minor sequence modifications (such as codon replacements, correction of mutated/deleted nucleotides) can be exploited to introduce precise genetic modifications at target loci. In contrast to NHEJ-mediated DSB repair, HDR-mediated repair is relatively inefficient and largely restricted to the S-phase of cycling cells. Recent reports demonstrate that HDR efficiency can be increased by inhibiting key molecules of the NHEJ pathway^{13, 14} or through timely delivery CRISPR/Cas9 during S-phase of the cell cycle¹⁵. Transient inhibition of Ku70 or Ligase IV, via shRNA knockdown, small-molecule inhibition, or proteolytic degradation increased HDR in HEK293, NIH3T3 and Burkitt lymphoma cells lines^{13, 14} Importantly, the impact that such treatments may have on Cas9 off-target activity remains to be investigated. Indeed, Ku70 deficiency results in growth retardation and leaky SCID phenotype¹⁶, whereas genetic ablation of Ligase IV causes late embryonic lethality and impaired V(D)J recombination in mice¹⁷. Ligase IV mutations in humans manifests as LIG4 Syndrome in which patients exhibit immunodeficiency and developmental/growth delay¹⁸. So far, there exists a bias as these studies have been done in proliferating cells that frequently enter into S-phase of the cell cycle. For post-mitotic cell types, such approaches may not be viable as components of HDR are missing. Moreover, inhibition of NHEJ may also impose risks for quiescent cells, such as hematopoietic stem cells, as they utilize the NHEJ pathway to repair accumulated DNA damage upon entry into cell cycle¹⁹.

There exists promise in timely delivery of CRISPR/Cas9 during S-phase¹⁵. This, unfortunately, is complicated by the difficulties in cell synchronization. Many agents used for arresting the cells in various phases of the cell cycle are toxic and may induce DNA damage. In an effort to bypass these limitations, however, we hypothesized that manipulation the DNA repair pathway choice and priming the cells for HDR through ectopic expression of components of HR pathway could increase the HDR efficiency irrespective of cell cycle status.

It is described herein that ectopic expression of Rad52 and dominant negative 53BP1 (dn53BP1) increases the HDR efficiency by more than 2.5-fold, resulting in a robust gene correction at the broken GFP locus in a reporter cell line. These findings were extended to multiple genetic loci and cell-types including human induced pluripotent stem (iPS) cells. Furthermore, using high throughput genome-wide translocation sequencing (HTGTS)²⁰, an unbiased off-target analysis approach revealed that ectopic expression of Rad52 and/or dn53BP1 does not adversely affect the Cas9-associated on-target specificity, off-target activity, or lead to increased wide-spread DSB. The present data indicate that this approach can very efficiently correct the disease-specific mutations in relevant primary cell types for therapeutic purposes.

Results

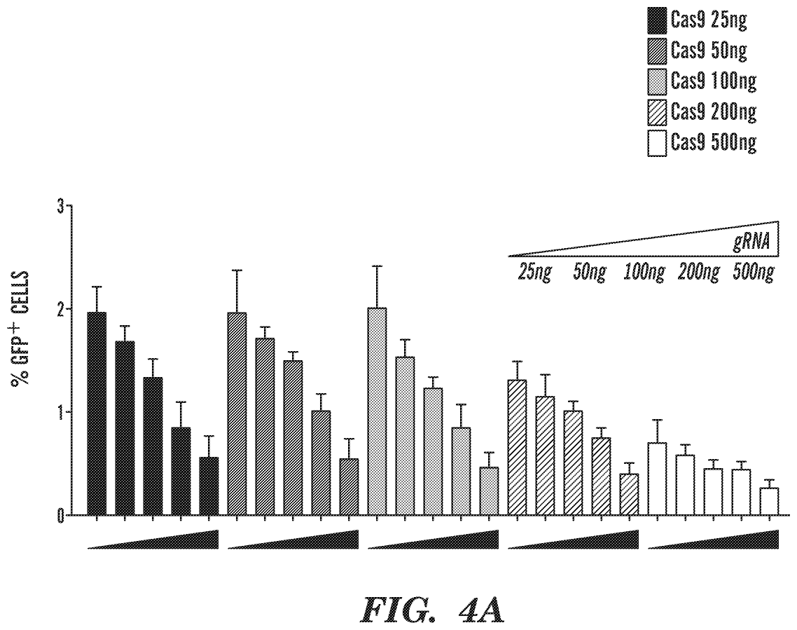

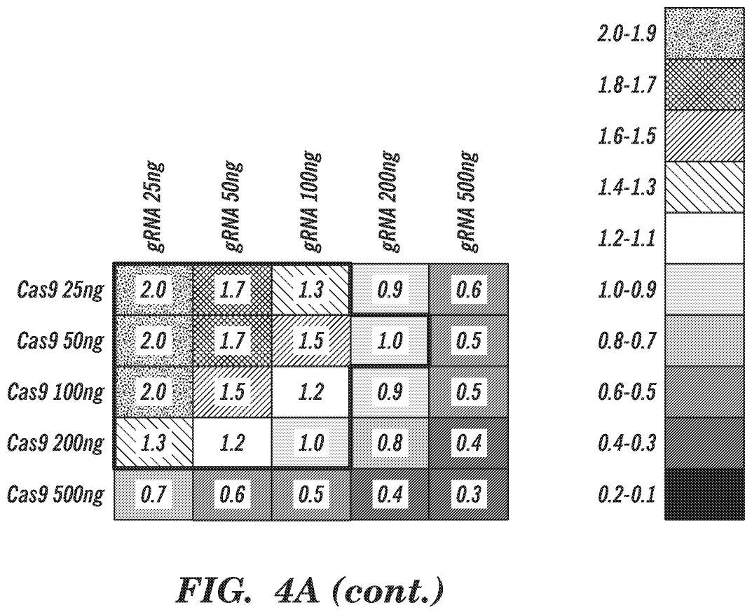

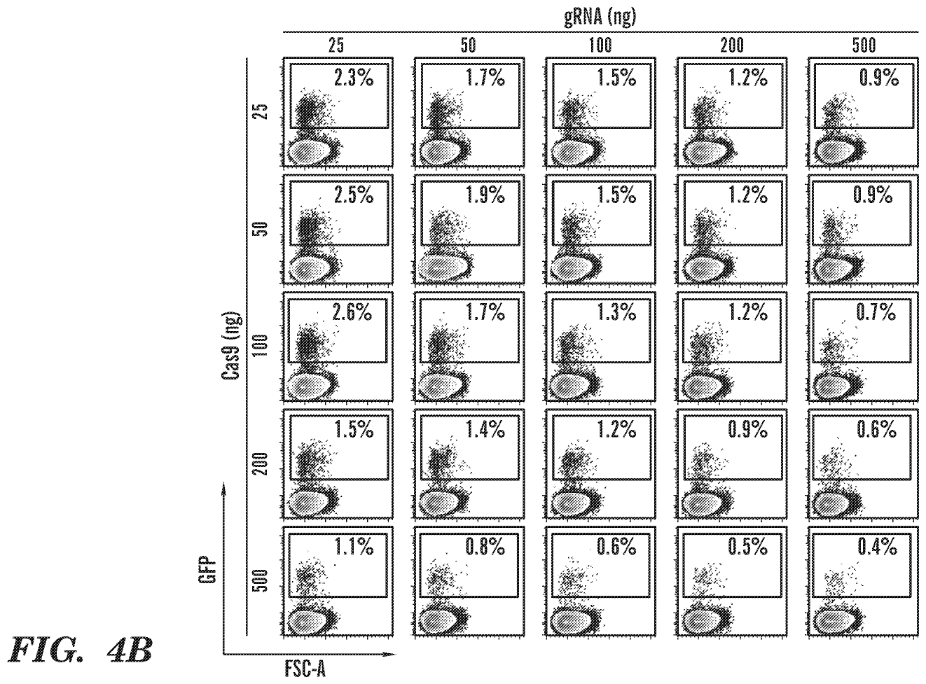

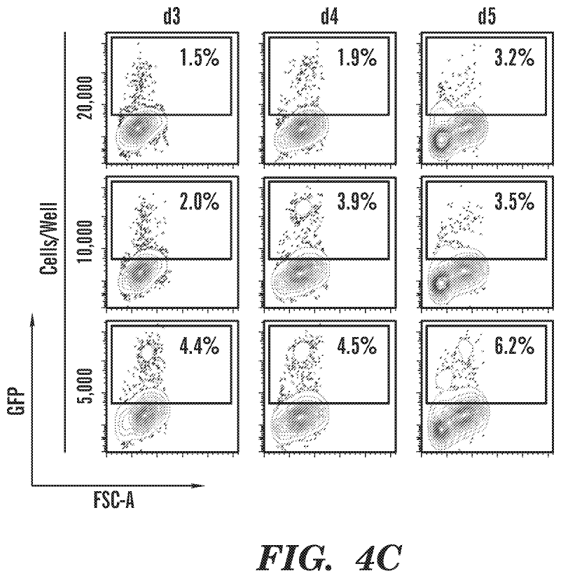

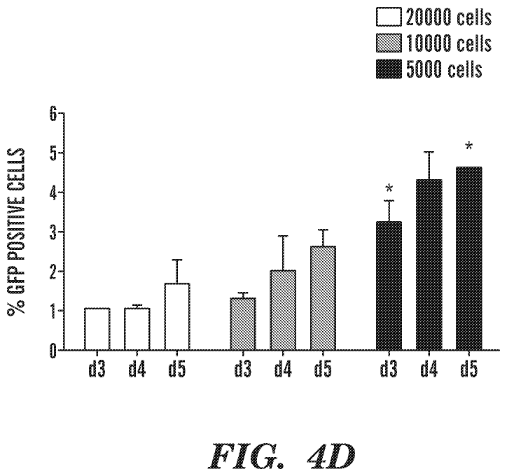

DNA repair pathway choice is largely determined by the cell cycle status—NHEJ in G0/G1 phase and HDR in S/G2/M phase of cell cycle. As the HDR is constrained by S/G2/M time window, it was reasoned that optimal delivery of Cas9, gRNA and donor template could be critical to HDR efficiency. In order to determine the optimal condition for robust HDR we used an established human HEK293 reporter cell line that gives a simple read-out of HDR by the repair of broken GFP sequences inserted in the genome⁴. Using this cell line, the amount of Cas9 and guide RNA (gRNA) (FIG. 4A, 4B), the number of days in between transfection and analysis (FIG. 4C, 4D), and concentration, length and orientation of the donor template (FIG. 5A-5C) were optimized. By transfecting 5,000 reporter cells (HEK293) with 25ng of each Cas9 and gRNA expression plasmids together with 4 pmol of (+)ssODN donor/repair template (184 bp, 92 nucleotide sequence homology on either side), it was demonstrated that the basal HDR efficiency could be improved to 15% (15.73%±0.40) (FIG. 5C, bottom panel) as measured by GFP expression.

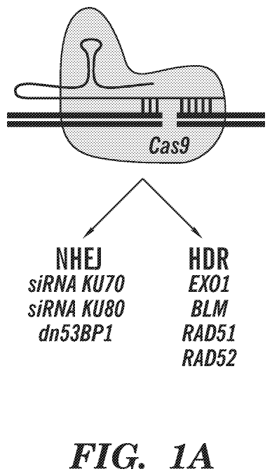

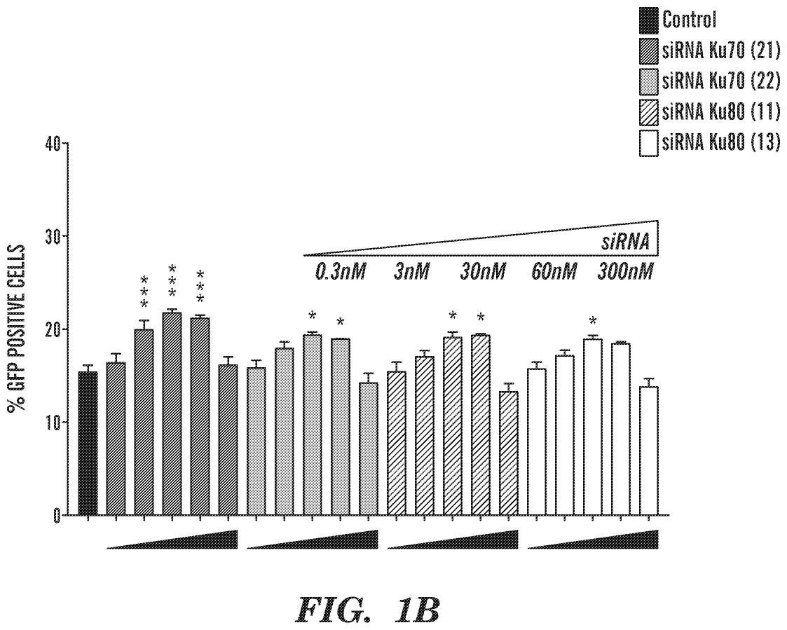

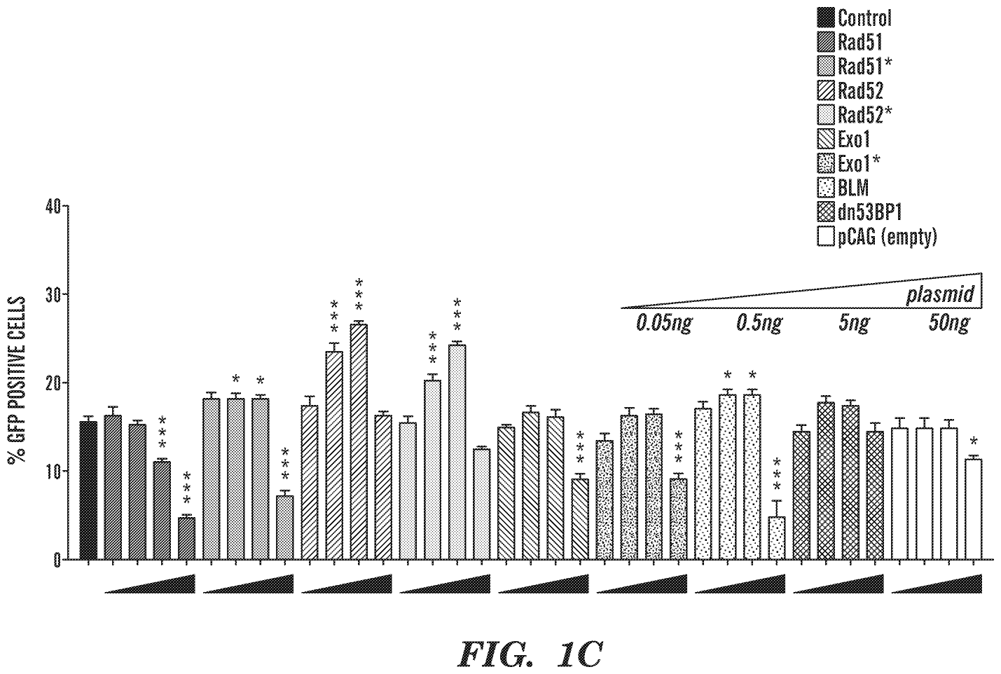

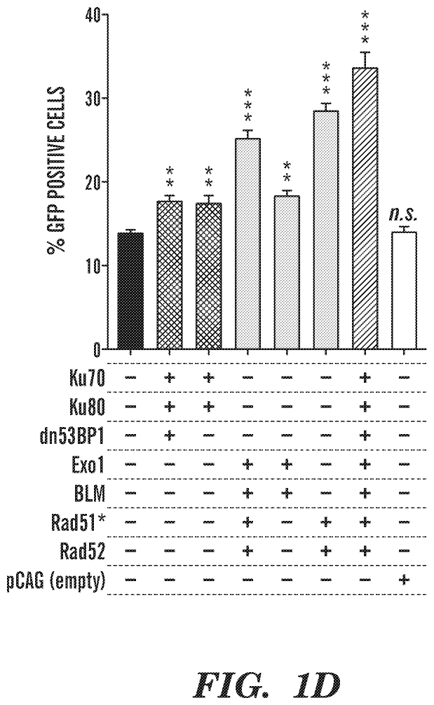

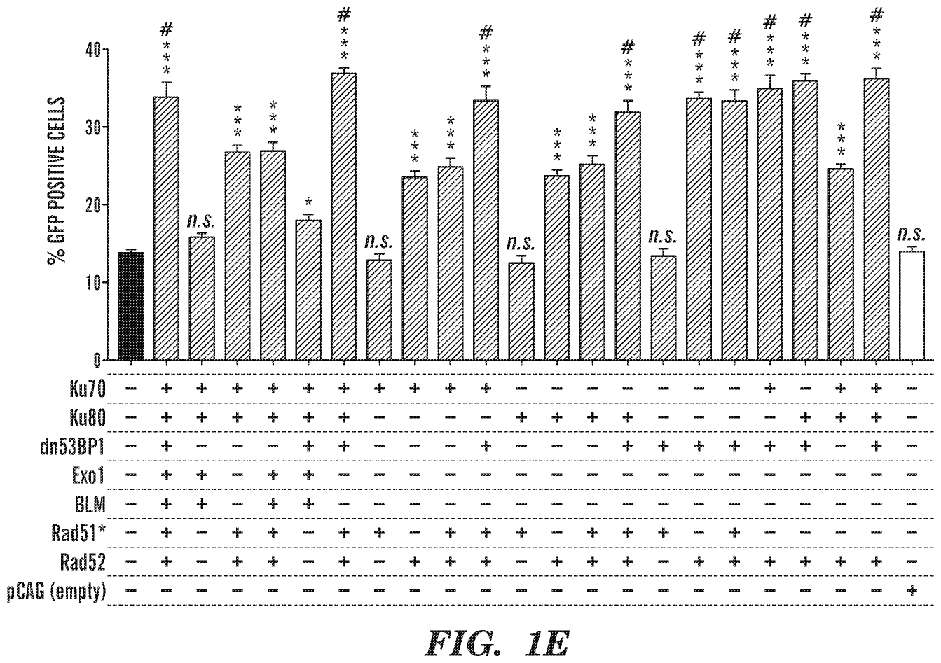

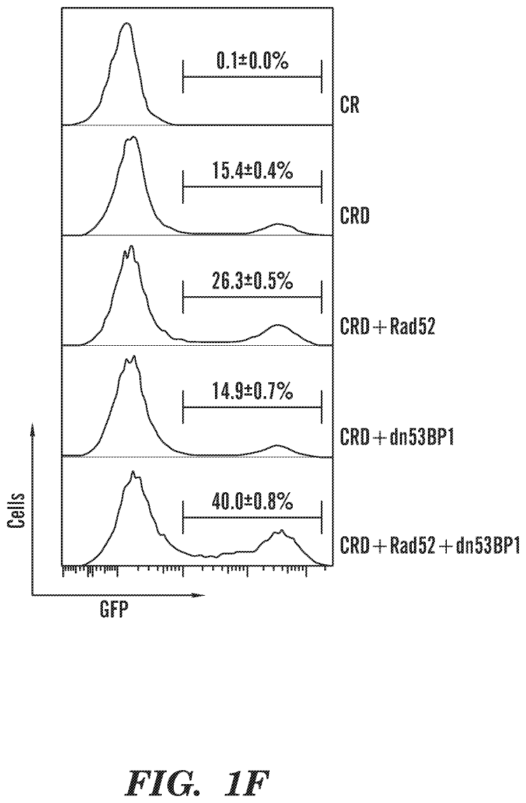

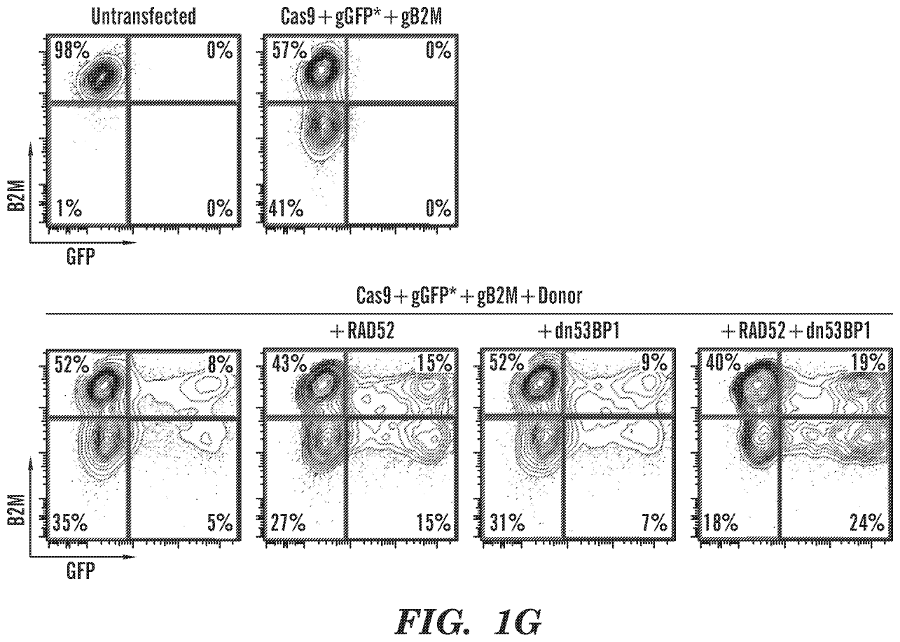

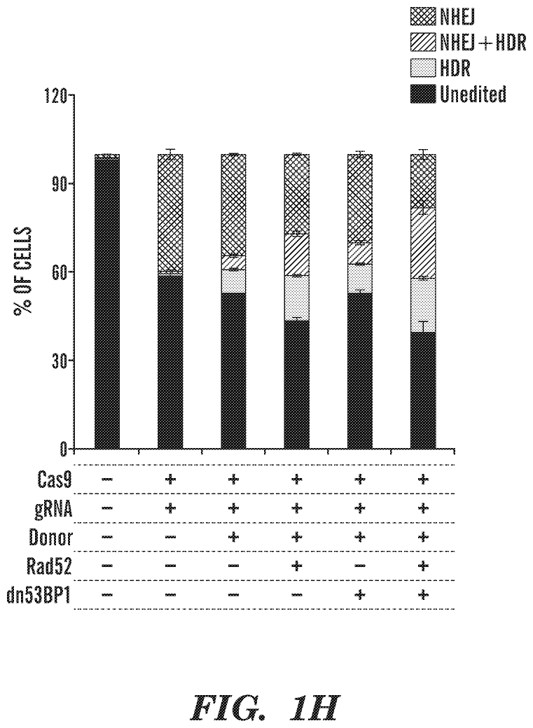

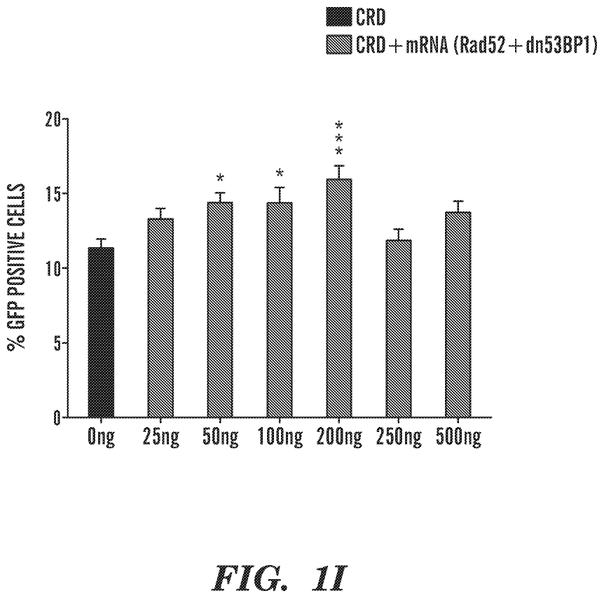

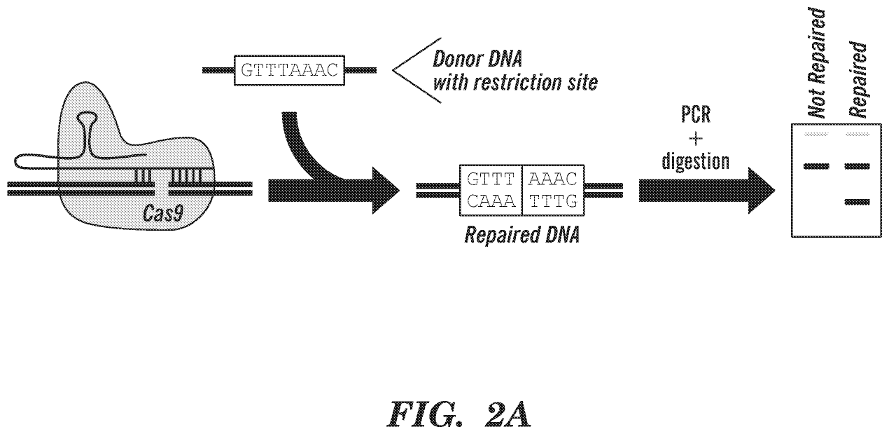

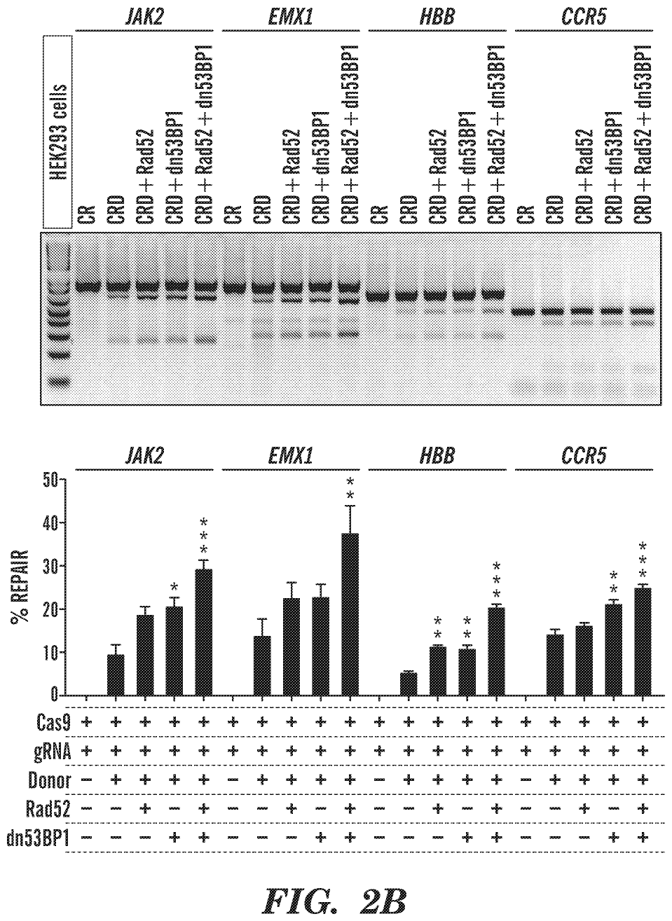

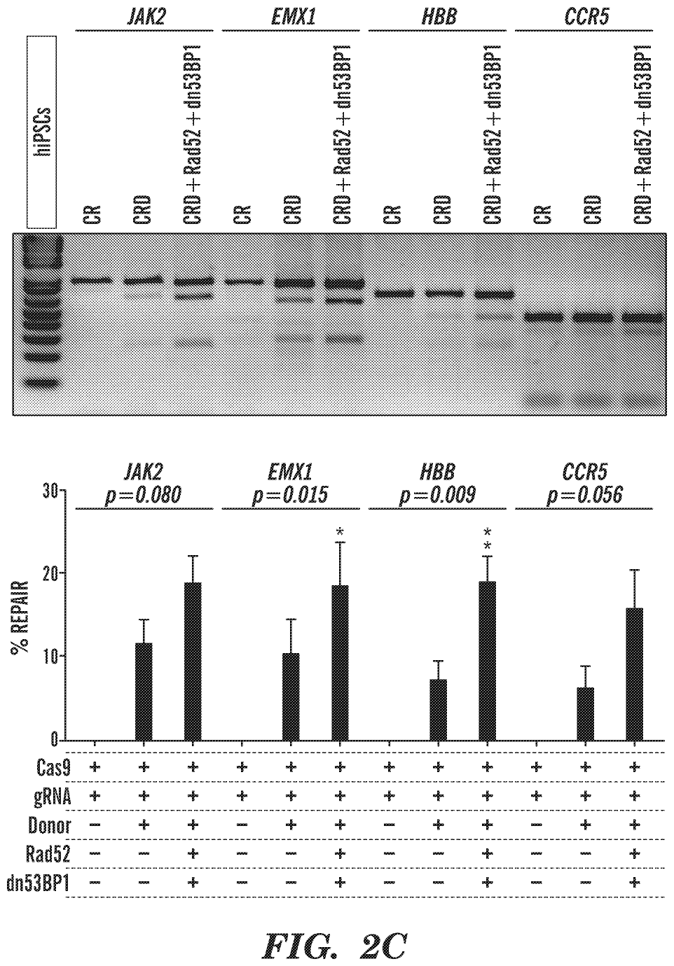

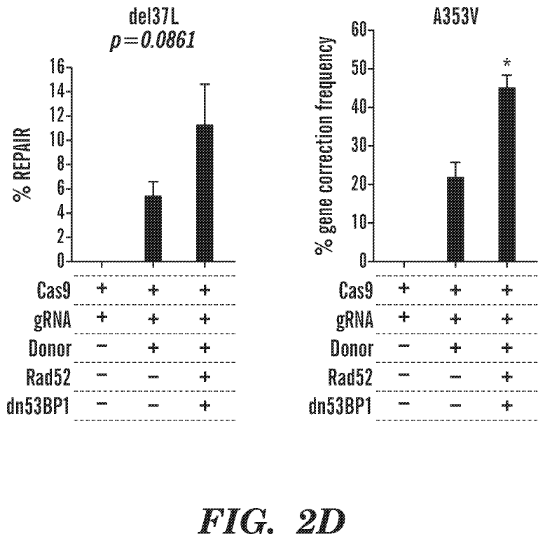

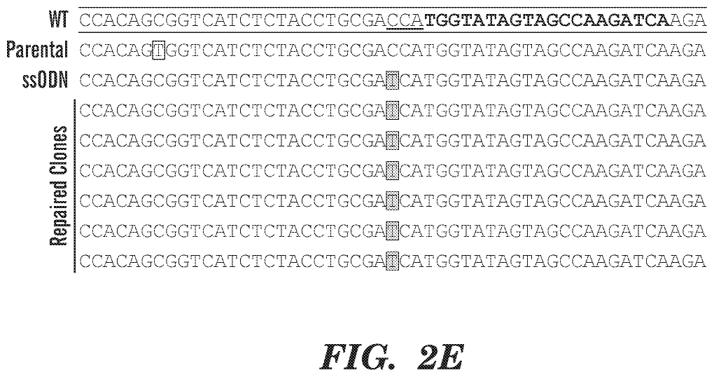

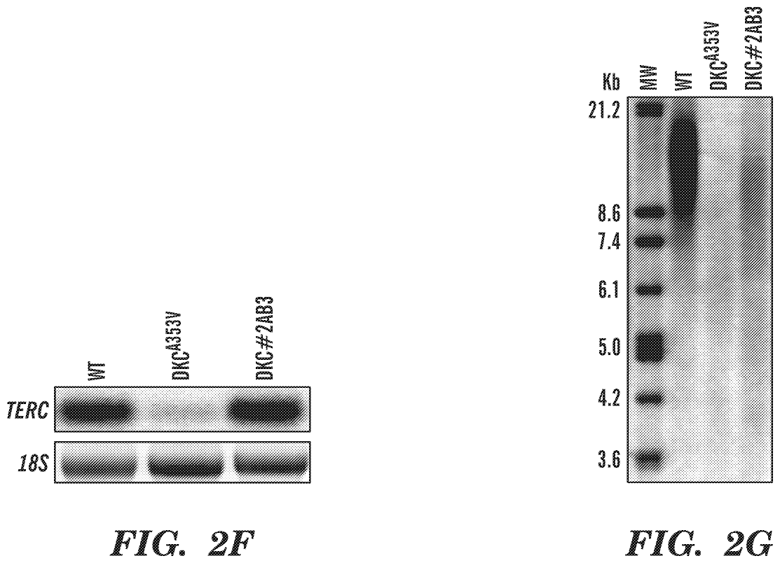

After the initial optimization, critical regulators of DNA repair pathway choice were tested (FIG. 1A). Ku70/80 play an important role in for NHEJ-mediated DNA repair. It has been reported that knocking down Ku70/80 improves HDR efficiency. It was found that siRNA-mediated knockdown of Ku70 or Ku80 marginally improved the HDR efficiency to 21.80%±0.42 (siRNAKu70), and 19.33%±0.23 (siRNAKu80) (FIG. 1B). As the expression of components of the HDR pathway is restricted to the S/G2/M phase of cell cycle, it was hypothesized that ectopic expression of key components of the HDR pathway would serve to improve the HDR. EXO1, BLM, RAD51, RAD52 and corresponding phosphomutants (EXO1^{S714E}, RAD51^{S309E}, RAD52^{Y104E} all denoted with an asterisks) were ectopically expressed together with Cas9, gRNA and donor template. Also included was a dominant negative form of 53BP1 (dn53BP1), containing solely the tandem Tudor domain to counteract with the function of 53BP1²¹, as this protein is implicated in XRCC4-dependent NHEJ and its inhibition has been reported to improve HDR efficiency²¹. The overexpression of RAD51, EXO1, EXO1* or dn53BP1 had a negligible impact on HDR efficiency, whereas RAD51* (18.10%±0.63) and BLM (18.67%±0.68) marginally increased the HDR efficiency compared to the control (15.38%±0.75) (FIG. 1C). Overexpression of either RAD52 (26.49%±0.53) or RAD52*, however, (24.26%±0.36) significantly improved HDR efficiency (FIG. 2C). This initial screen demonstrates that either knockdown of Ku70/80 or overexpression of RAD52, RAD51*, BLM, Exo1 improves HDR efficiency. Subsequently, the combination of factors was tested, based on the reasoning that if they act in additive/synergetic manner, it will further improve the HDR efficiency. Towards this end, these factors were assayed in various combinations. It was first tested whether the factors that could potentially block NHEJ (Ku70/80 and dn53BP1, red bars, FIG. 1D). The second combination consisted of factors involved in HDR (EXO1, BLM, RAD51* and RAD52, green bars, FIG. 1D). In third combination we co-expressed all the factors together (blue bar, FIG. 1D). Though all three combinations showed significant increase in GFP⁺ cells, the most robust induction of HDR was achieved with all factors combined together (33.70%+1.83) (FIG. 1D), suggesting that suppressing NHEJ together with ectopic expression of components of HR pathway complement each other in a synergistic fashion to improve HDR efficiency (FIG. 1D).

By testing different combinations of these components, 9 different conditions were identified in which the percentage of GFP⁺ cells were comparable and improved the HDR by more than 2.5-fold higher than the control (FIG. 1E). Out of these conditions, the simplest one was the combination of RAD52 and dn53BP1 (33.55%±0.84 compared to control, 13.81%±0.48) (FIG. 1E-1F). Interestingly, RAD52 and dn53BP1 were present in all other 8 conditions. This data indicates that RAD52 and dn53BP1 are both necessary and sufficient for improving HDR efficiency in lieu of the other components.

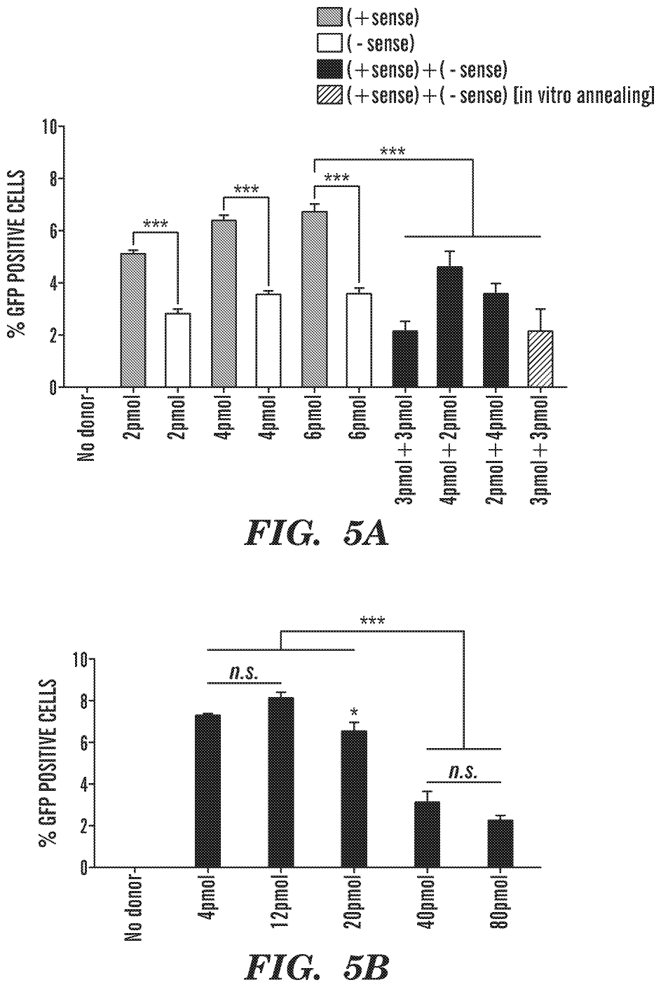

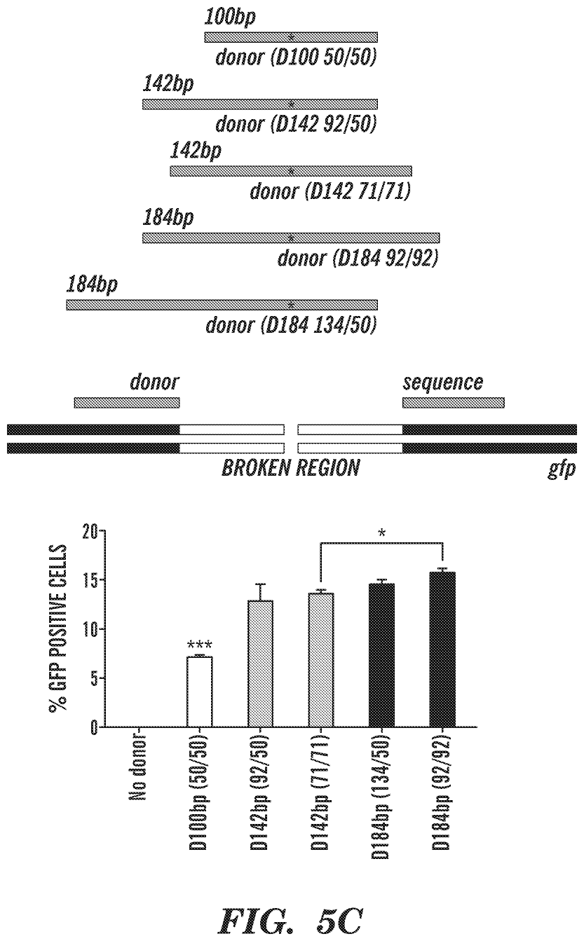

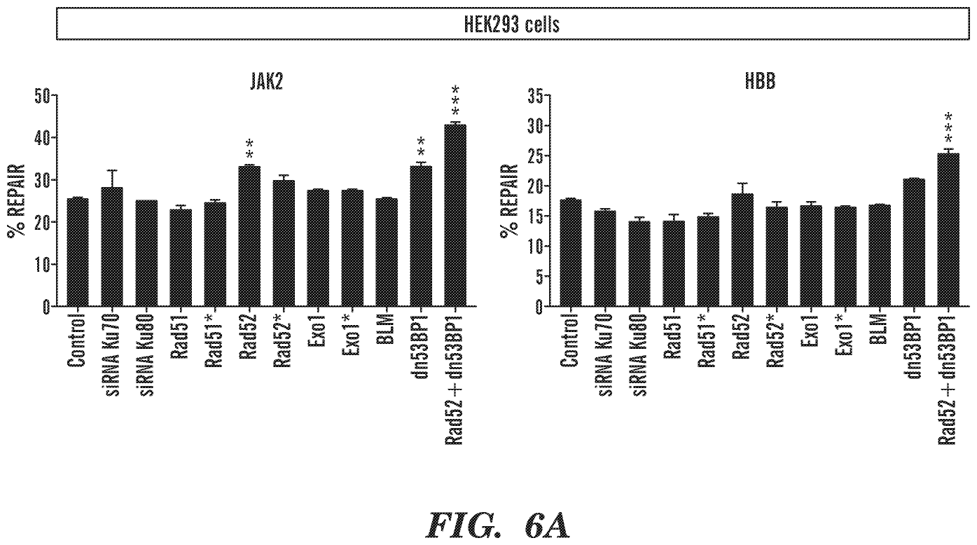

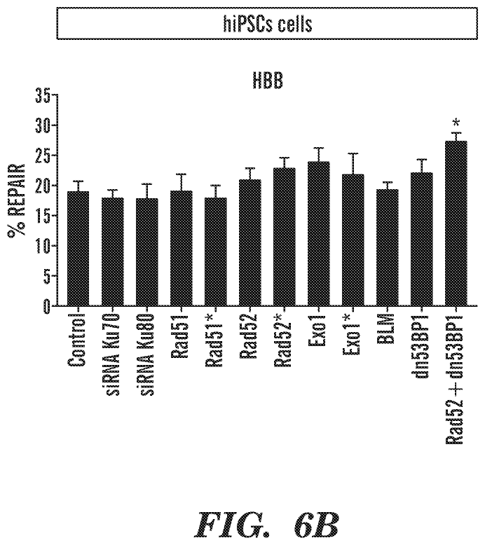

To validate the robustness and applicability of this approach, several clinical relevant genetic loci (JAK2, EMX1, HBB, and CCR5) were targeted in HEK293 cells. In order to verify HDR activity, a restriction endonuclease (PmeI) recognition sequence (GTTTAAAC) was knocked-in at the targeted loci (FIG. 2A), such that HDR efficiency can be calculated easily by restriction digestion of the PCR amplicon generated from the amplification of the target gene. Consistent with the observation with broken GFP cell line, the co-expression of RAD52 and dn53BP1 resulted in improved HDR efficiency at all the targeted loci (FIG. 2B) indicating that precise genomic modification can be achieved using this approach. However, in contrast to broken GFP reporter cell line, dn53BP1 alone resulted in improved HDR at various loci in HEK293 (FIG. 2B) with comparable or even greater HDR efficiency than RAD52 alone. As such contrasting result may originate from multiple integration of broken GFP cassette in the genome or genomic structure of targeted locus. Therefore each candidate gene was tested individually in HEK293 cells (FIG. 6A-6B) and RAD52, dn53BP1 or combination of RAD52 and dn53BP1 were identified as superior over other candidate genes in improving HDR efficiency.

Next, these findings were extended to human iPS cells by targeting above-mentioned loci (JAK2, EMX1, HBB, and CCR5) by knocking-in PmeI restriction site at the targeted loci and analyzed the HDR efficiency by PmeI restriction digestion. As expected, the results confirmed that ectopic expression of RAD52 and dn53BP1 also increased the HDR efficiency by at least 2-fold for all targeted loci (FIG. 2C). To test whether the present approach can be used to restore gene function by repairing disease-causing mutation, the technology described herein was used to correct mutation resulting in X-linked Dyskeratosis Congenita. Patient-derived iPS lines (harboring the del37L deletion or A353V mutation in Dyskerin 1 gene (DKC1)²² were taken in to consideration such that repair of the DKC1 mutations (del37L or A353V) will respectively restore XmnI or MspA1I restriction endonuclease site that could be detected and quantified by restriction digestion analysis (FIG. 2D). After transfecting the iPS lines with all the CRISPR components and donor template, iPS cells were transiently selected over Puromycin for 36 hours and cells were clonally expanded and each clone was analyzed by PCR and restriction digestion. For del37L lines, we got 5.3±1.2% of repaired clones in the control, compared to 11.2±3.4% of repaired clones in the condition where RAD52 and dn53BP1 were added (FIG. 2D). Similar results were obtained for the A353V iPS cells line in which 1.9±0.9% was achieved in the control and 8.4±0.9% in the condition with both RAD52 and dn53BP1 (FIG. 2D). Sanger sequencing was performed on corrected clones to confirm the correction of disease-causing mutations (FIG. 2E).

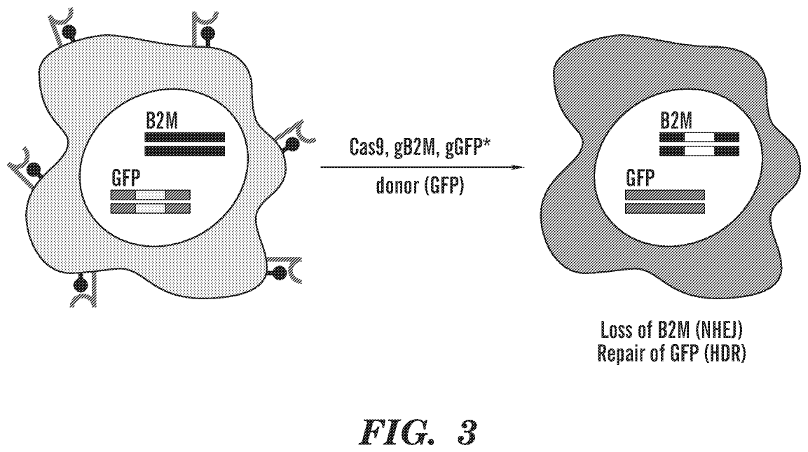

To gain insight into the mechanism behind the increased HDR efficiency in presence of RAD52 and dn53BP1, a cellular system was developed that monitors both NHEJ and HDR simultaneously and independently by fluorescent-based analysis. Addition of gRNA-targeting accessory chain β2-microglobin (B2M) allowed measurement of NHEJ activity by monitoring loss of B2M expression in the broken GFP HDR-reporter system (FIG. 3A, 3B). Upon transfection of Cas9, gRNA targeting broken-GFP and B2M (gGFP* and gB2M), and donor template for GFP, the majority of cells lost B2M expression (39.07±0.25%) indicating robust induction of NHEJ activity, whereas approximately 6% (6.06±0.25%) of cells were exclusively GFP⁺, demonstrating HDR activity. A fraction of cells, however, approximately 4% (3.93±0.17%) were GFP⁺ and B2M-suggesting that a small percentage of B2M⁻ cells (NHEJ competent) were capable of undergoing HDR (FIG. 3C). Notably, in the presence of RAD52 and dn53BP1, there was a 5-fold increase in B2M⁻GFP⁺ cells (23.13±1.60% vs 3.93±0.17%), indicating that ectopic expression of these factors imbues HDR potential on an NHEJ competent cell (FIG. 3C). On the other hand, there was only a 2-fold increase in exclusively GFP⁺ cells (14.40±0.30% vs 6.06±0.25%). Unlike earlier studies, upon transient expression of RAD52 and dn53BP1 NHEJ potential remained largely unaffected.

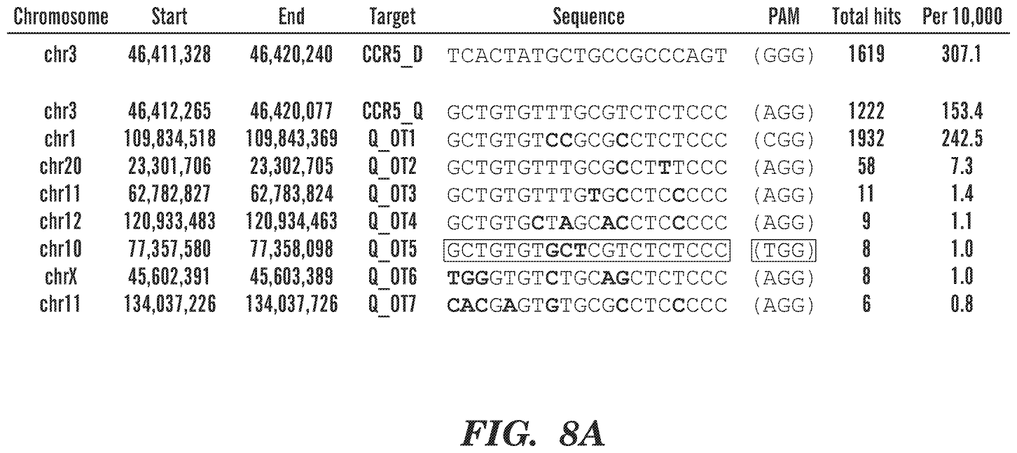

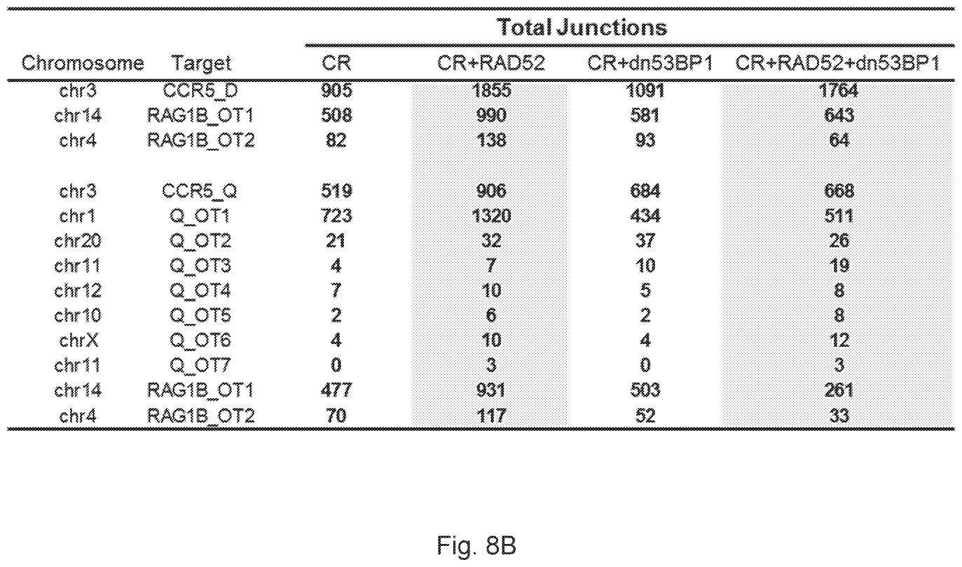

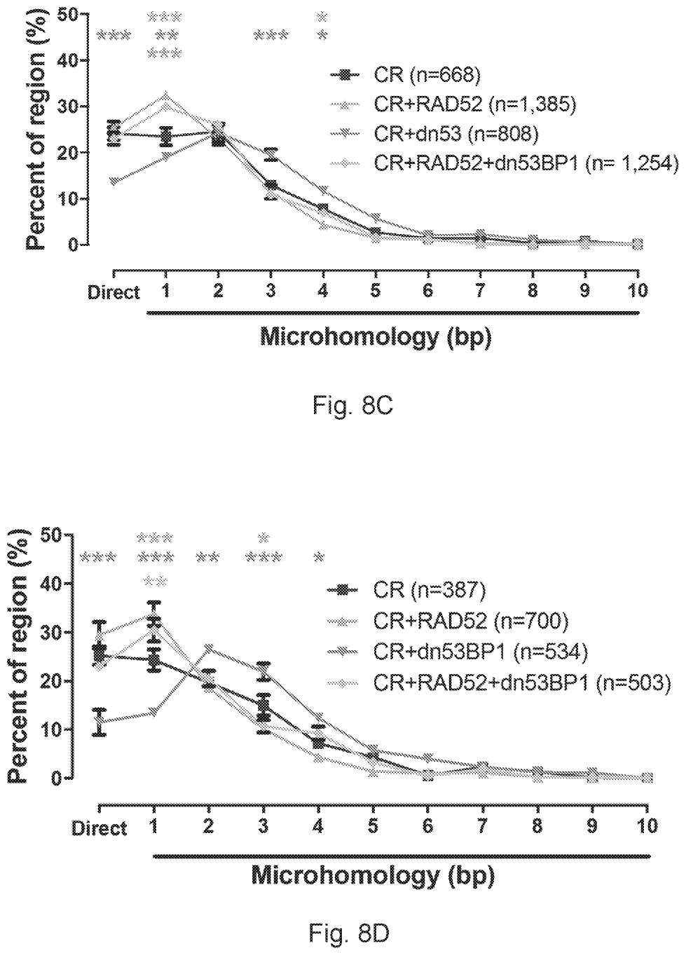

The present data indicates that ectopic expression of RAD52 and dn53BP1 improves HDR efficiency without compromising NHEJ-mediated DNA repair. However the impact of DNA repair pathway choice manipulation on genomic integrity is largely unaddressed. To address the impact of ectopic expression of RAD52 and dn53BP1 genomic integrity and CRISPR/Cas9-associated off-target effects the state of the art “High Throughput Genome-wide Translocation Sequencing” (HTGTS)²⁰—an unbiased approach to monitor off-target (OT) cutting—was utilized. Algorithm-based off-target prediction has demonstrated that out of 5 gRNA targeting CCR5 one gRNA (crCCR5B) resulted in significant off-target cutting activity at one particular locus (CCR2) in human primary CD34+ hematopoietic stem and progenitor cells (HSPC) due to sequence homology and intact PAM motif¹¹. As CRISPR/Cas9 may have unintended cutting activity at totally unrelated sequences that can not be predicted by an algorithm, the off-target activity of each guide by HTGTS was examined (data not shown). Though majority of the OTs identified by HGTS overlapped with those identified previously, it was possible to detect novel OTs (data not shown). To study the impact of RAD52 and dn53BP1 on OTs and genomic integrity one guide with no OTs (CCR5D) and other guide that has 7 OTs identified by HTGTS (CCR5Q) were further examined. HTGTS analysis showed that there were no adverse impact of ectopic expression of RAD52 and dn53BP1 on specificity of CRISPR/Cas9 as there was no increase in number of OTs for two gRNA targeting CCR5 (FIG. 8B).

Discussion

The CRISPR/Cas9 system allows genetic manipulation of mammalian cells with unprecedented efficacy and accuracy. DSB created by the Cas9 nuclease are largely repaired by the error-prone NHEJ repair pathway, which generates InDels at the break site resulting in gene ablation. A battery of knockout human cell lines and mouse models has been generated using CRISPR/Cas9. This system is proving to be an indispensable resource for forward genetics and drug discovery. Though very efficacious ablation of genes of clinical relevance in primary human hematopoietic cells has been achieved using the CRISPR/Cas9 system¹¹, efficient repair of disease causing mutation in relevant primary cells has not been achieved so far due to extremely infrequent utilization of HDR. It was hypothesized that by manipulating DNA repair choice HDR efficiency could be improved. Through the optimization of the delivery and expression of all key components of CRISPR/Cas9 system and donor templates, it is demonstreated herein that the basal HDR activity can be improved up to 10-15% (FIG. 4A-4D). In addition, key components that can manipulate the DNA repair pathways choice, either by blocking NHEJ and/or inducing HDR, were screened for. As described herein, suppression of Ku70/80 resulted in marginal increases in HDR efficiency. Through transient ectopic expression of key regulators of DNA repair pathways (RAD51, RAD52, dn53BP1, EXO1, BLM), it was demonstrated that by manipulating the DNA repair pathway choice, HDR efficiency could be improved. Transient ectopic expression of RAD52 and dn53BP1 leads to a 2-3-fold increase in HDR efficiency compared to basal level. Without wishing to be bound by theory, though 9 different combinations of key regulators of DNA repair pathway that increase the HDR efficiency are identified herein, (FIG. 1A-1F), the presence of RAD52 and dn53BP1 in all combinations suggests that ectopic expression of RAD52 and dn53BP1 are both necessary and sufficient to improve the HDR efficiency. Even the transient ectopic expression of RAD52 and dn53BP1 by mod-mRNA was able to improve the HDR efficiency comparable to plasmid-based expression. This straightforward approach demonstrated that precisely defined genetic modifications can be achieved at targeted loci. 4 different loci (CCR5, JAK2, HBB, and DM were targeted by knocking-in a restriction endonuclease recognition sequence that shows the applicability and robustness of the approach describe herein. The methods and compostions described herein can permit efficient repair of genetic lesions and also facilitate the creation of precise genetic modifications such as codon alterations or knocking-in a reporter cassette. Finally, the findings were validating by correcting a disease-specific mutation in Dyskeratosis Congenita patient-derived iPS cells. The methods and compositions described herein can permit correction of disease-specific mutations in primary stem and progenitor cells and can bring the CRISPR/Cas9 technology to therapeutic genome engineering and clinical translation.

Materials and Methods

Generation of CRISPR/Cas9 Vectors, gRNAs, Candidate Genes Plasmids, siRNAs and Modified mRNAs

A human-codon-optimized Cas9 gene with a C-terminal nuclear localization signal⁴ was subcloned into a CAG expression plasmid. PX459 plasmid encoding Cas9 and puromycin was purchased from Addgene. gRNA targeting the GFP sequence⁴ was cloned in a plasmid with the human U6 polymerase III promoter. All the other gRNA sequences published earlier were cloned into the pGuide plasmid using BbsI restriction sites⁴.

Plasmids encoding components of the DNA repair pathways (RAD51, RAD52, Exo1, BLM and dn53BP1) were obtained from Harvard PlasmID Database. PCR products of the genes were then subcloned into a CAG expression plasmid and sequenced. The phosphomutant versions (Exo1*: S714E; Rad51*: S309E; Rad52*: Y104E) were generated by site-directed mutagenesis.

siRNAs for Ku70 and Ku80 were purchased from Sigma-Aldrich

Modified mRNAs were generated as described by Mandal et al.

Cell Culture

HEK293.

HEK293 cells were maintained in DMEM (Gibco) supplied with 10% FBS (Gibco) and Penicillin-Streptomycin (Gibco). Cells were passaged two times per week with trypsin (Gibco). SCR7 inhibitor [1 μM] was added (Xcess Biosciences, San Diego, USA) 12 hours after transfection and remained until analysis—after 72 hours^{13, 14}.

Human iPS Cells.

iPS lines (BJ RiPS and DK patient-derived iPS cells) were described previously^{22, 23}. iPS cells were maintained onto hESC-qualified Matrigel (BD Biosciences) in mTeSR (Stem Cell Technologies). For transfection, cells were maintained onto Matrix and Pluripro (both from CELL guidance systems) and enzymatic passaging was done with TrypLE (Life Tehnologies) and Rock inhibitor [Y-27632, 10 μM] (Calbiochem). For all the conditions, media were changed daily and cells were split once a week.

Transfection of Cells

Plasmids.

HEK293 cells were seeded in 96-well plates the day before transfection (5,000 cells/well). On the day of transfection, cells were transfected with plasmids (Cas9: 25ng/well; gRNA: 25ng/well; donor template: 4 pmol/well; Rad52 and dn53BP1: 5ng/well—unless specified) mixed with Opti-MEM (Invitrogen) and 0.3 μl/well (optimized value) of Trans-IT 293 reagent (Mirus) according to the manufacturer's recommendation. After 15 minutes of incubation at room temperature, 9 μl the mix was dropped slowly into each well. Cells were analyzed 96 hours after transfection.

iPS cells were plated in 48-well plates the day before transfection (30,000 cells/well) in Pluripro (Cell Guidance). After 24 hours, plasmids (Cas9-puro: 62.5ng/well; gRNA: 62.5ng/well; donor template: 10 pmol/well; Rad52 and dn53BP1: 12.5ng/well) were mixed with Opti-MEM and 0.780/well Trans-IT LT1 (Mirus) according to the manufacturer's datasheet. After 15 minutes of incubation at room temperature, 26 μl of the mix was dropped slowly into each well. For antibiotics selection, 1 μg/ml of puromycin was added after 24 hours of transfection for 2 days. After 72 hours of transfection, iPS cells were enzymatically detached and plated onto matrigel and mTeSR in a ratio of 5,000 cells/60 mm dish. 10 μM of Y-27632 was added to increase single cell survival after passaging. When the colonies started to appear, each clone was manually collected and split into 2 wells of a 96-well plate with matrigel and mTeSR1 (Stem Cell Technologies). One of the wells was reserved to do clonal screening by PCR and the other well was to start clonal expansion.

Modified mRNAs.

Modified mRNA transfections were carried out in HEK293 cells 24 hours after transfection of plasmids, as specified previously. Modified mRNAs (100 ng/well) were diluted in 7.50 Stemfect Buffer (Stemgent) as recommended by manufacturer and mixed with 0.60/well of Stemfect Reagent (Stemgent) plus 7.5 μl Stemfect Buffer. After 15 minutes of incubation at room temperature, 50 μl of HEK293 growth medium were added to the mix and immediately dropped into each well. After 24 hours, medium was changed to avoid toxicity. Cells were analyzed 96 hours after plasmid transfection.

Surveyor Assay.

PCR products of each targeted genes were amplified by PCR using the Phusion polymerase and HF Buffer (New England Biolabs). CEL assay was performed by using the Surveyor Mutation detection kit (Integrated DNA Technologies) as recommended by manufacturer's instructions.

Flow Cytometry.

For flow cytometry analysis, cells were trypsinized and resuspended in sample medium (1×PBS without Ca²⁺ and Mg²⁻, 2% FBS, 2 mM EDTA). Cells were incubated with human anti-B2M-APC as described earlier¹¹. Propidium Iodite (Sigma-Aldrich) 1-2 μg/ml was added to the cells prior to analysis to exclude dead cells. Analyses were done at a FACSCanto™ machine (BD). FACS data were analyzed using FlowJo™ software.

HTGTS and Off-Target Analysis. Off-target analysis was carried out by HTGTS as described earlier²⁰. Briefly, HEK293 cells were co-transfected with expression plasmids encoding Cas9:RAG1B (20 μg), guide RNA targeting CCR5 (10 μg), RAD52 and dn53BP1 (5 μg each) and GFA (5 μg) using Calcium Phosphate. Cells were lysed 48 hours post-transfection and DNA was isolated by ethanol precipitation. 50 μg of DNA was processed for HTGTS as described²⁰.

### REFERENCES

1. Jinek, M. et al. A programmable dual-RNA-guided DNA endonuclease in adaptive bacterial immunity. Science 337, 816-821 (2012).
2. Cong, L. et al. Multiplex genome engineering using CRISPR/Cas systems. Science 339, 819-823 (2013).
3. Jinek, M. et al. RNA-programmed genome editing in human cells. eLife 2, e00471 (2013).
4. Mali, P. et al. RNA-guided human genome engineering via Cas9. Science 339, 823-826 (2013).
5. Hsu, P. D., Lander, E. S. & Zhang, F. Development and applications of CRISPR-Cas9 for genome engineering. Cell 157, 1262-1278 (2014).
6. Sander, J. D. & Joung, J. K. CRISPR-Cas systems for editing, regulating and targeting genomes. Nature biotechnology 32, 347-355 (2014).
7. Ding, Q. et al Enhanced efficiency of human pluripotent stem cell genome editing through replacing TALENs with CRISPRs. Cell stem cell 12, 393-394 (2013).
8. Gilbert, L. A. et al. Genome-Scale CRISPR-Mediated Control of Gene Repression and Activation. Cell 159, 647-661 (2014).
9. Hruscha, A. et al. Efficient CRISPR/Cas9 genome editing with low off-target effects in zebrafish. Development 140, 4982-4987 (2013).
10. Li, D. et al. Heritable gene targeting in the mouse and rat using a CRISPR-Cas system. Nature biotechnology 31, 681-683 (2013).
11. Mandal, P. K. et al. Efficient ablation of genes in human hematopoietic stem and effector cells using CRISPR/Cas9. Cell stem cell 15, 643-652 (2014).
12. Ran, F. A. et al. In vivo genome editing using Staphylococcus aureus Cas9. Nature 520, 186-191 (2015).
13. Maruyama, T. et al. Increasing the efficiency of precise genome editing with CRISPR-Cas9 by inhibition of nonhomologous end joining. Nature biotechnology 33, 538-542 (2015).
14. Chu, V. T. et al. Increasing the efficiency of homology-directed repair for CRISPR-Cas9-induced precise gene editing in mammalian cells. Nature biotechnology 33, 543-548 (2015).
15. Lin, S., Staahl, B. T., Alla, R. K. & Doudna, J. A. Enhanced homology-directed human genome engineering by controlled timing of CRISPR/Cas9 delivery. eLife 4 (2014).
16. Gu, Y. et al. Growth retardation and leaky SCID phenotype of Ku70-deficient mice. Immunity 7, 653-665 (1997).
17. Frank, K. M. et al. Late embryonic lethality and impaired V(D)J recombination in mice lacking DNA ligase IV. Nature 396, 173-177 (1998).
18. O'Driscoll, M. et al. DNA ligase IV mutations identified in patients exhibiting developmental delay and immunodeficiency. Mol Cell 8, 1175-1185 (2001).
19. Beerman, I., Seita, J., Inlay, M. A., Weissman, I. L. & Rossi, D. J. Quiescent Hematopoietic Stem Cells Accumulate DNA Damage during Aging that Is Repaired upon Entry into Cell Cycle. Cell stem cell (2014).
20. Frock, R. L. et al. Genome-wide detection of DNA double-stranded breaks induced by engineered nucleases. Nature biotechnology (2014).
21. Xie, A. et al. Distinct roles of chromatin-associated proteins MDC1 and 53BP1 in mammalian double-strand break repair. Mol Cell 28, 1045-1057 (2007).
22. Agarwal, S. et al. Telomere elongation in induced pluripotent stem cells from dyskeratosis congenita patients. Nature 464, 292-296 (2010).
23. Warren, L. et al. Highly efficient reprogramming to pluripotency and directed differentiation of human cells with synthetic modified mRNA. Cell stem cell 7, 618-630 (2010).

### Example 2: Manipulation of DNA Damage Repair Pathway Choice Improves Homology-Directed Repair During CRISPR/Cas9-Mediated Genome Editing

Summary.

Gene disruption by CRISPR/Cas9 is highly efficient and generally relies on the error-prone non-homologous end joining (NHEJ) pathway. Precise gene editing, however, requires the presence of a donor DNA template and engagement of the homology-directed DNA repair (HDR) pathway, which occurs at reduced frequency in most mammalian cells. The inventors hypothesized that manipulation of DNA damage response pathway choice might be an effective strategy to improve HDR efficacy. As described herein, key factors involved in both NHEJ and homologous recombination (HR) repair pathways were screened. Transient ectopic expression of RAD52 and a dominant-negative form of 53BP1 (dn53BP1) engenders HDR-mediated gene editing efficacies in up to 40% of human cells. High throughput genome-wide translocation sequencing revealed that expression of RAD52 and dn53BP1 does not alter CRISPR/Cas9 specificity. These data show that manipulation of DNA repair pathway choice is an effective strategy for bringing precision genome editing towards clinical application.

Introduction.

Repurposing type II bacterial CRISPR system as a genome-editing tool (Jinek et al., 2012) has provided a robust technology for site-directed genome editing in mammalian cells (Cong et al., 2013; Hsu et al., 2014; Jinek et al., 2013; Mali et al., 2013) including at disease relevant loci in primary cells (Hendel et al., 2015; Mandal et al., 2014). Double strand breaks (DSBs) generated by Cas9, followed by NHEJ-mediated repair frequently results in small insertions and deletions disrupting coding or regulatory sequences. Alternatively, DSBs can be repaired through the homology-directed repair (HDR) pathway in the presence of a donor DNA template. HDR however, is restricted to the S/G2-phase of cycling cells (Hufnagl et al., 2015; Kakarougkas and Jeggo, 2014; Karanam et al., 2012) and is utilized, on average, an order of magnitude less than NHEJ-mediated DSB repair in many cell types (Mao et al., 2008). This has limited the utility of CRISPR/Cas9 and other programmable nucleases for applications requiring precise genome modification such as correction of mutations underlying genetic disease (Hsu et al., 2014). To overcome this limitation, recent studies have taken diverse approaches including timely delivery of CRISPR/Cas9 during S-phase of the cell cycle (Lin et al., 2014) or inhibiting key molecules of the NHEJ pathway (Chu et al., 2015; Maruyama et al., 2015; Robert et al., 2015). However, caveats to such approaches include the impracticality of cell synchronization particularly for in vivo settings, whereas inhibition of NHEJ may have adverse consequences on genome stability as suggested by genetic studies (Ferguson et al., 2000; Frank et al., 1998; Gu et al., 1997; O'Driscoll et al., 2001). As demonstrated herein, manipulation of components of both HR and NHEJ can be used to modulate repair pathway choice towards increased utilization of HDR.

Results

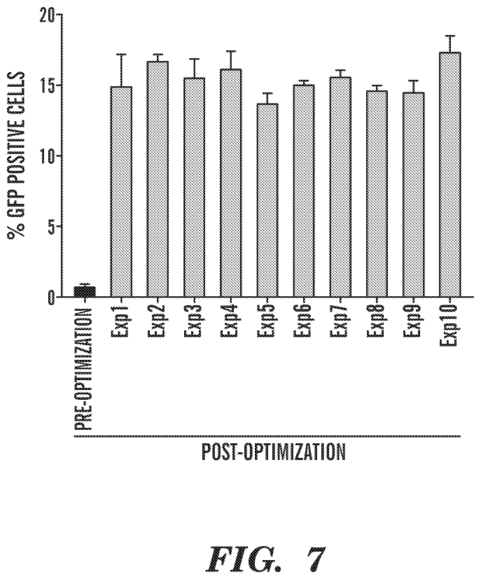

Towards the goal of improving HDR, an established human HEK293 reporter cell line in which HDR frequency can be assessed by repair of a broken GFP cassette was used (Mali et al., 2013). Cas9 and guide RNA (gRNA) concentration were optimized (FIG. 4A-4B), cell density (FIG. 4C-4D), and concentration, length and orientation of the donor template (FIG. 5A-5C) and achieved a robust and reproducible basal HDR frequency of 15.7±0.4% (FIG. 7).

Cells lacking components of NHEJ show a propensity toward increased HDR efficiency (Pierce et al., 2001) and consistent with this, inhibition of NHEJ components (Ku, Ligase IV, or DNA-PKc) via shRNA knockdown, proteolytic degradation or pharmacological inhibition have recently been shown to improve HDR frequency (Chu et al., 2015; Maruyama et al., 2015; Robert et al., 2015). In agreement with published reports, it is demonstrated herein that siRNA knockdown of Ku70 or Ku80 significantly improved HDR frequency above baseline (FIG. 1B). As utilization and reliance on different DNA repair pathways is cell cycle regulated (Branzei and Foiani, 2008), and further that homologous recombination (HR) is predominantly utilized in the S/G2 phase of the cell cycle, it was hypothesized that inhibition of NHEJ alone might not be sufficient to achieve maximal HDR activity. RAD51, RAD52, EXO1, BLM, along with mutant versions of RAD51 (RAD51^{S309E}) (Sorensen et al., 2005), RAD52 (RAD52^{Y104E}) (Honda et al.; 2011), and EXO1 (EXO1^{S714E}) (Bolderson et al., 2010) were ectopically expressed. Also expressed was a dominant negative form of 53BP1 (dn53BP1), containing only the tandem Tudor domain that has been reported to improve HDR efficiency by counteracting the function of 53BP1 in XRCC4-dependent NHEJ (Xie et al., 2007). Overexpression of RAD51, EXO1, EXO1^{S714E} or dn53BP1 had no impact on HDR, whereas RAD51^{S309E} (18.1±0.6%) and BLM (18.7±0.7%) marginally increased the HDR efficiency compared to controls (15.4±0.8%) (FIG. 1C). In contrast, overexpression of either RAD52 (26.5±0.5%) or RAD52^{Y104E} (24.3±0.4%) appreciably improved HDR frequency (FIG. 1C).

To determine if these factors act in a synergistic manner to further improve HDR, a combinatorial approach was used net. The factors that could potentially inhibit NHEJ (siRNAs for Ku70/80 and dn53BP1), augment HR (EXO1, BLM, RAD51^{S309E} and RAD52), and a combination of all the factors together were tested (FIG. 1D). Though all combinations showed significant increase in HDR, the highest frequency was achieved when all factors were combined (33.7±1.8%), suggesting that inhibition of NHEJ and augmentation of HR synergize to increase HDR (FIG. 1D). In order to determine the minimal combination of factors sufficient to maximize HDR the combinations of factors involved in HR and NHEJ were further stratified and 9 different conditions were identified that showed comparably robust HDR (FIG. 1E). Strikingly, RAD52 and dn53BP1 were present in all these conditions, and indeed co-expression of RAD52 and dn53BP1 alone was sufficient to achieve maximal HDR (FIGS. 1E and 1F). To explore the impact of RAD52 and dn53BP1 on HDR and NHEJ independently, a system allowing simultaneous monitoring of NHEJ and HDR at different loci was developed, in which a gRNA targeting the MHC-I accessory chain β2-microglobin (B2M) provides a measure of NHEJ (Mandal et al., 2014), and repair of broken GFP (FIG. 1A-1F) indicates HDR frequency. Upon transfection of Cas9, gRNAs targeting broken-GFP and B2M (gGFP* and gB2M), and donor template for GFP, exclusive loss of B2M expression in 39.1±0.3% of cells indicating robust NHEJ was observed, whereas 6.1±0.3% of the cells were exclusively GFP⁺, indicative of HDR. A smaller fraction of cells (3.9±0.2%) were GFP⁺ and B2M⁻ indicating that a minor fraction of cells had undergone both NHEJ and HDR (FIGS. 1G and 1H). As expected, in the presence of RAD52, HDR frequency was significantly elevated; dn53BP1 alone did not improve HDR, whereas the maximal increase in HDR was observed when both proteins were co-expressed. Interestingly, loss of B2M remained remarkably constant in all treatments indicating that NHEJ activity remained unaffected by expression of RAD52 and dn53BP1 (FIG. 1H).

To test the robustness and applicability of this approach at other loci and other cell types, several clinically relevant genes (JAK2, EMX1, HBB, and CCR5) were targeted in HEK293 cells and human induced pluripotent stem (iPS) cells. In order to monitor HDR activity, donor templates containing a PmeI recognition sequence (GTTTAAAC) were designed for each locus (FIG. 2A). Consistent with the previous observations (FIG. 1F-1H), co-expression of RAD52 and dn53BP1 resulted in significantly improved HDR efficiency at all of the targeted loci in both cell types demonstrating broad applicability of this approach (FIG. 2B-2C). However, surprisingly, in contrast to repair of the broken GFP (FIG. 1C, 1F, and 1G), dn53BP1 alone resulted in improved HDR at 3 out of 4 loci targeted in HEK293 with comparable or even greater HDR efficiency than RAD52 alone (FIG. 2B). This prompted rescreening of all of the original candidate factors monitoring HDR at JAK2 and HBB in HEK293 cells and HBB in iPS cells, where it was found that in all cases, co-expression of RAD52 and dn53BP1 maximally increased HDR frequency (data not shown).

The efficacy through which a disease-causing mutation could be corrected by applying the present technology was tested by repairing a mutation in the DKC1 gene underlying X-linked dyskeratosis congenita in a patient-derived iPS cell line. Correction of the most frequently recurrent disease-associated mutation, c.1058C>T which results in the amino acid change p.A353V, restores an MspA1l restriction site to the locus, which allows an estimation of HDR frequency upon restriction digestion analysis. Co-expression of RAD52 and dn53BP1 resulted in a correction frequency of 45.0±3.4% compared to control (21.6±4.1%) (FIG. 2D). Targeted iPS cells were clonally expanded and Sanger sequencing confirmed correction of mutated base in all of the clones that yielded PCR amplicons that could be digested with MspA1I. Dyskerin plays a crucial role in telomere maintenance by stabilization of the telomerase RNA component (TERC) (Mitchell et al., 1999). Dyskerin activity was assayed on a corrected clone (DKC1#2AB3) in comparison to wild-type and parental DKC1^{A353V} controls by measuring TERC levels by Northern blot (FIG. 2F), and telomere length by Southern blot (FIG. 2G). The corrected clone showed TERC levels comparable to wild-type iPS cells (FIG. 2F), and concomitant elongation of telomere length (FIG. 2G) compared to the parental cell line.

The data described herein, along with previous studies (Chu et al., 2015; Maruyama et al., 2015: Robert et al., 2015) focused on suppressing NHEJ demonstrate that manipulation of DNA repair pathways can be an effective strategy for improving HDR. However, the impact that such manipulations may have on genomic integrity has not been addressed. To address the impact of ectopic expression of RAD52 and dn53BP1 on genomic integrity and CRISPR/Cas9 specificity High Throughput Genome-wide Translocation Sequencing (HTGTS) was utilized (Frock et al., 2014)—an unbiased approach to monitor the specificity and off-target (OT) activity of Cas9 and other engineered nucleases as well as other types of DSBs. For this purpose, two gRNAs were used, targeting the chemokine receptor CCR5 (gCCR5D and gCCR5Q) that did not exhibit OT activity in primary human CD34⁺ hematopoietic stem and progenitor cell as determined by algorithm-based OT prediction and targeted deep sequencing analysis in a previous study (Mandal et al., 2014). Consistent with the foregoing findings, gCCR5D only exhibited on-target activity in HEK293 cells with no OT (FIGS. 8A-8D). Interestingly however, HTGTS revealed that gCCR5Q exhibited significant OT activity at 7 genomic sites that were previously predicted and interrogated in CD34⁺ HSPCs by target capture deep sequencing (Table 3) (Mandal et al., 2014). Importantly, ectopic co-expression of RAD52 and dn53BP1 did not alter the specificity of CRISPR/Cas9 as no change in OT activity was observed (FIGS. 8A-8D). Furthermore, HTGTS did not detect substantially increased widespread, low-level DSB activity when RAD52 and dn53BP1 were co-expressed.

Discussion

The CRISPR/Cas9 system allows genetic manipulation of mammalian cells with unprecedented ease and efficacy particularly for applications in which gene disruption is the desired outcome. However, harnessing the full potential of CRISPR/Cas9 for precision gene editing including repair of disease-causing mutations is currently limited by infrequent utilization of HDR due to the intrinsic cell cycle properties of different cell types in which the S/G2 phase of the cell cycle is either short (eg. most mitotic cells), rarely engaged (eg. quiescent stem cells), or inaccessible (eg. post-mitotic cells). Moreover, NHEJ is also active in S/G2 and competes with the HR pathway for DSB repair (Karanam et al., 2012). It was therefore hypothesized that manipulation of DNA repair pathway choice by augmenting HR and/or suppressing NHEJ could be effective means of increasing HDR utilization. Indeed by screening key regulators of DNA repair it was identified herein that co-expression of, e.g., RAD52 and dn53BP1 significantly improves HDR frequency at multiple loci in human cells, including the correction of a disease-causing mutation in patient-derived iPS cells. Whether or not RAD52 and dn53BP1 increases HDR by enhancing HR pathway utilization during S/G2, or rather imbues HDR potential onto cells in other phases of the cell cycle is currently unclear. Nonetheless, the fact that NHEJ activity was unaltered by expression of RAD52 and dn53BP1 (FIGS. 1G and 1H) indicates that NHEJ inhibition was not the primary driver of the elevated HDR we observed upon expression of these proteins.

Previous efforts towards improving HDR by modulating DNA repair have mainly focused on transiently suppressing NHEJ by a number of strategies (Chu et al., 2015; Maruyama et al., 2015; Robert et al., 2015) though the consequences that such manipulations may have on genome integrity were unaddressed. Importantly, NHEJ pathway integrity is critical for maintaining genome stability and cells lacking NHEJ display gross genomic abnormalities and are prone to chromosomal translocations (Ferguson et al., 2000). Ku70 deficiency results in growth retardation and a leaky SCID phenotype (Gu et al., 1997), and genetic ablation of Ligase IV causes late embryonic lethality in mice (Frank et al., 1998), whereas Ligase IV mutations in humans manifests as LIG4 Syndrome in which patients exhibit immunodeficiency and developmental/growth delay (O'Driscoll et al., 2001). We therefore addressed this issue in our study using HTGTS and found that expression of RAD52 and/or dn53BP1 neither altered CRISPR/Cas9 specificity (eg. on-, off-target), nor led to detectable changes in recurrent widespread DSB activity, suggesting that genomic integrity was not dramatically affected. Taken together, the data presented herein indicate that manipulation of DNA repair pathway choice is a powerful strategy for overcoming a critical bottleneck to exploiting the full potential of CRISPR/Cas9 for precision genome editing.

### REFERENCES

Bolderson, E., Tomimatsu, N., Richard, D. J., Boucher, D., Kumar, R., Pandita, T. K., Burma, S., and Khanna, K. K. (2010). Phosphorylation of Exo1 modulates homologous recombination repair of DNA double-strand breaks. Nucleic acids research 38, 1821-1831.
Branzei, D., and Foiani, M. (2008). Regulation of DNA repair throughout the cell cycle. Nature reviews Molecular cell biology 9, 297-308.
Chu, V. T., Weber, T., Wefers, B., Wurst, W., Sander, S., Rajewsky, K., and Kuhn, R. (2015). Increasing the efficiency of homology-directed repair for CRISPR-Cas9-induced precise gene editing in mammalian cells. Nature biotechnology 33, 543-548.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
Ferguson, D. O., Sekiguchi, J. M., Chang, S., Frank, K. M., Gao, Y., DePinho, R. A., and Alt, F. W. (2000). The nonhomologous end-joining pathway of DNA repair is required for genomic stability and the suppression of translocations. Proceedings of the National Academy of Sciences of the United States of America 97, 6630-6633.

Frank, K. M., Sekiguchi, J. M., Seidl, K. J., Swat, W., Rathbun, G. A., Cheng, H. L., Davidson, L., Kangaloo, L., and Alt, F. W. (1998). Late embryonic lethality and impaired V(D)J recombination in mice lacking DNA ligase IV. Nature 396, 173-177.
Frock, R. L., Hu, J., Meyers, R. M., Ho, Y. J., Kii, E., and Alt, F. W. (2014). Genome-wide detection of DNA double-stranded breaks induced by engineered nucleases. Nature biotechnology.
Gu, Y., Seidl, K. J., Rathbun, G. A., Zhu, C., Manis, J. P., van der Stoep, N., Davidson, L., Cheng, H. L., Sekiguchi, J. M., Frank, K., et al. (1997). Growth retardation and leaky SCID phenotype of Ku70-deficient mice. Immunity 7, 653-665.
Hendel, A., Bak, R. O., Clark, J. T., Kennedy, A. B., Ryan, D. E., Roy, S., Steinfeld, I., Lunstad, B. D., Kaiser, R. J., Wilkens, A. B., et al. (2015). Chemically modified guide RNAs enhance CRISPR-Cas genome editing in human primary cells. Nature biotechnology 33, 985-989.
Honda, M., Okuno, Y., Yoo, J., Ha. T., and Spies, M. (2011). Tyrosine phosphorylation enhances RAD52-mediated annealing by modulating its DNA binding. EMBO J 30, 3368-3382.
Hsu, P. D., Lander, E. S., and Zhang, F. (2014). Development and applications of CRISPR-Cas9 for genome engineering. Cell 157, 1262-1278.
Hufnagl, A., Herr, L., Friedrich, T., Durante, M., Taucher-Scholz, G., and Scholz, M. (2015). The link between cell-cycle dependent radiosensitivity and repair pathways: a model based on the local, sister-chromatid conformation dependent switch between NHEJ and HR. DNA Repair (Amst) 27, 28-39.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., East, A., Cheng, A., Lin, S., Ma, E., and Doudna, J. (2013). RNA-programmed genome editing in human cells. eLife 2, e00471.
Kakarougkas, A., and Jeggo, P. A. (2014). DNA DSB repair pathway choice: an orchestrated handover mechanism. Br J Radiol 87, 20130685.
Karanam, K., Kafri, R., Loewer, A., and Lahav, G. (2012). Quantitative live cell imaging reveals a gradual shift between DNA repair mechanisms and a maximal use of HR in mid S phase. Mol Cell 47, 320-329.
Lin, S., Staahl, B. T., Alla, R. K., and Doudna, J. A. (2014) Enhanced homology-directed human genome engineering by controlled timing of CRISPR/Cas9 delivery. eLife 4.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Mandal, P. K., Ferreira, L. M., Collins, R., Meissner, T. B., Boutwell, C. L., Friesen, M., Vrbanac, V., Garrison, B. S., Stortchevoi, A., Bryder, D., et al. (2014). Efficient ablation of genes in human hematopoietic stem and effector cells using CRISPR/Cas9. Cell stem cell 15, 643-652.
Mao, Z., Bozzella, M., Seluanov, A., and Gorbunova, V. (2008). Comparison of nonhomologous end joining and homologous recombination in human cells. DNA Repair (Amst) 7, 1765-1771.
Maruyama, T., Dougan, S. K., Truttmann, M. C., Bilate, A. M., Ingram, J. R., and Ploegh, H. L. (2015). Increasing the efficiency of precise genome editing with CRISPR-Cas9 by inhibition of nonhomologous end joining. Nature biotechnology 33, 538-542.
Mitchell, J. R., Wood, E., and Collins, K. (1999). A telomerase component is defective in the human disease dyskeratosis congenita. Nature 402, 551-555.
O'Driscoll, M., Cerosaletti, K. M., Girard, P. M., Dai, Y., Stumm, M., Kysela, B., Hirsch, B., Gennery, A., Palmer, S. E., Seidel, J., et al. (2001). DNA ligase IV mutations identified in patients exhibiting developmental delay and immunodeficiency. Mol Cell 8, 1175-1185.
Pierce, A. J., Hu, P., Han, M., Ellis, N., and Jasin, M. (2001). Ku DNA end-binding protein modulates homologous repair of double-strand breaks in mammalian cells. Genes Dev 15, 3237-3242.
Robert, F., Barbeau, M., Ethier, S., Dostie, J., and Pelletier, J. (2015). Pharmacological inhibition of DNA-PK stimulates Cas9-mediated genome editing. Genome Med 7, 93.
Sorensen, C. S., Hansen, L. T., Dziegielewski, J., Syljuasen, R. G., Lundin, C., Bartek, J., and Helleday, T. (2005). The cell-cycle checkpoint kinase Chkl is required for mammalian homologous recombination repair. Nat Cell Biol 7, 195-201.
Xie, A., Hartlerode, A., Stucki, M., Odate, S., Puget, N., Kwok, A., Nagaraju, G., Yan, C., Alt, F. W., Chen, J., et al. (2007). Distinct roles of chromatin-associated proteins MDC1 and 53BP1 in mammalian double-strand break repair. Mol Cell 28, 1045-1057.

TABLE 3
List of guide RNAs, primers and donor templates. Table 3 discloses SEQ ID NOS 14-38,
respectively, in order of appearance.

| | | | | |
|---|---|---|---|---|
| Gene | Spacer Sequence | Reference | PCR primers | Donor Template |

| | | | | |
|---|---|---|---|---|
| AAVS1* | GGGCCACTAGGGACAGGAT | Mali et al. | NA | TGAAGCAGCACGACTTCTTCAAGTCCGCC |
| | | 2013 | | ATGCCCGAAGGCTACGTCCAGGAGCGCAC |
| | | | | CATCTTCTTCAAGGACGACGGCAACTACA |
| | | | | AGACCCGCGCCGAGGTGAAGTTCGAGGG |
| | | | | CGACACCCTGGTGAACCGCATCGAGCTGA |
| | | | | AGGGCATCGACTTCAAGGAGGACGGCAA |
| | | | | CATCCTGGGGCA |
| JAK2 | AATTATGGAGTATGTGTCTG | Smith et al. | F: ACGTTGATGGCAGTTGCAGGTC | TTCCTTAGTCTTTCTTTGAAGCAGCAAGTA |
| | | 2015 | R: | TGATGAGCAAGCTTTCTCACAAGCATTTG |
| | | | CTGACAGAGTTGCTAGACACTGGGTT | GTTTTAAATTATGGAGTATGTGTgtttaaacCT |
| | | | G | GTGGAGACGAGAGTAAGTAAAACTACAG |
| | | | | GCTTTCTAATGCCTTTCTCAGAGCATCTGT |
| | | | | TTTTGTTTATATAGAAAATTCAGTTTCAGG |
| | | | | ATCA |
| EMX1 | GTCACCTCCAATGACTAGGG | Lin et al. | F: GTCTTCCCATCAGGCTCTCAGCTC | AAGAAGGGCTCCCATCACATCAACCGGTG |
| | | 2015 | R: GAGCTGGAGGTAGAGACCAGGGT | GCGCATTGCCACGAAGCAGGCCAATGGG |
| | | | | GAGGACATCGATGTCACCTCCAATGACTA |
| | | | | gtttaaacGGGTGGGCAACCACAAACCCACGA |
| | | | | GGGCAGAGTGCTGCTTGCTGCTGGCCAGG |
| | | | | CCCCTGCGTGGGCCCAAGCTGGACTCTGG |
| | | | | CCACTCCC |
| HBB | AGTCTGCCGGTTACTGCCCTG | Cardick et | F: TGCCAGAAGAGCCAAGGACAGGTA | ATTTGCTTCTGACACAACTGTGTTCACTAG |
| | | al. 2013 | R: CATCAAGCGTCCCATAGACTCACC | CAACCTCAAACAGACACCATGGTGCATCT |
| | | | | GACTCCTGAGGAGAAGTCTGCCGTTACTG |
| | | | | CCgtttaaacCTGTGGGGCAAGGTGAACGTGG |
| | | | | ATGAAGTTGGTGGTGAGGCCCTGGGCAGGn |
| | | | | TTGGTATCAAGGTTACAAGACAGGTTTAA |
| | | | | GGAGACCAAT |
| CCR5 | gCCR5D: | Mandal et | F: CTGCAAAAGGCTGAAGAGCA | CATGACTGACATCTACCTGCTCAACCTGG |
| | TCACTATGCTGCCGCCCAGT | al. 2014 | R: CCCCAAGATGACTATCTTTAATGTC | CCATCTCTGACCTGTTTTTCCTTCTTACTG |
| | | | | TCCCCTTCTGGGCTCACTATGCTGCCGCCg |
| | | | | tttaaacCAGTGGGACTTTGGAAATACAATGT |
| | | | | GTCAACTCTTGACAGGGCTCTATTTTATA |
| | | | | GGCTTCTTCTCTGGAATCTTCTTCATCATC |
| | | | | CTCCC |
| | gCCR5Q: | Mandal et | Same primer pair as above | GTCCATGCTGTGTTTGCTTTAAAAGCCAG |
| | GCTGTGTTTGCGTCTCTCCC | al. 2014 | | GACGGTCACCTTTGGGGTGGTGACAAGTG |
| | | | | TGATCACTTGGGTGGTGGCTGTGTTTGCG |
| | | | | TCTCTgtttaaacCCCAGGAATCATCTTTACCA |
| | | | | GATCTCAAAAAGAAGGTCTTCATTACACC |
| | | | | TGCAGCTCTCATTTTCCATACAGTCAGTAT |
| | | | | CAATTCTGGAAGA |
| B2M | GCTACTCTCTCTTTCTGGCC | Mandal et | NA | NA |
| | | al. 2014 | | |
| DKC1 | TGATCTTGGCTACTATACCA | Present | F: GAGCTGCAAGCCTGTTATGTG | GAATTGGGGCTCATCAATTATCAATTCTTT |
| | | Study | R: CGCAACCCAGTACCATTAC | CACCCTTCAAATAATTCTTTTCTTTATTCA |
| | | | | ATGCCTGTAGCTATTGCATTAATGACCAca |
| | | | | gcggTCATCTCTACCTGCGACCATGGTATA |
| | | | | GTAGCCAAGATCAAGAGAGTGATCATGG |
| | | | | AGAGAGACACTTACCCTCGGAAGTGGGGT |
| | | | | TTAGGTC |

### Example 3

Over-expression of RAD52 and/or dn53BP1 does not alter the specificity of Cas9. HTGTS was conducted to analyse off-target effects for gRNA targeting CCR5 (FIG. 8A). Comparison of the number of translocation junctions under control conditions or the presence of RAD52 and/or dn53BP1 overexpression found no significant change in off-target effects (FIG. 8B). However, analysis of translocation junctions with respect to the RAG1 universal bait revealed significant differences in microhomology distritution. RAD52 alone promoted increased end-joining with minimal (1 bp) microhomology over control transfected samples, whereas ectopic expression of dn53BP1 alone resulted in diminished direct end-joining and increased joining of DNA ends with greater microhomology distritutions (2-5 bp)(FIGS. 8C-8D). Upon co-expression of RAD52 and dn53BP1, the effect of RAD52 antagonized the impact of dn53BP1, favouring direct end-joining and end-joining with minimal (1 bp) microhomology (FIGS. 8C-D).

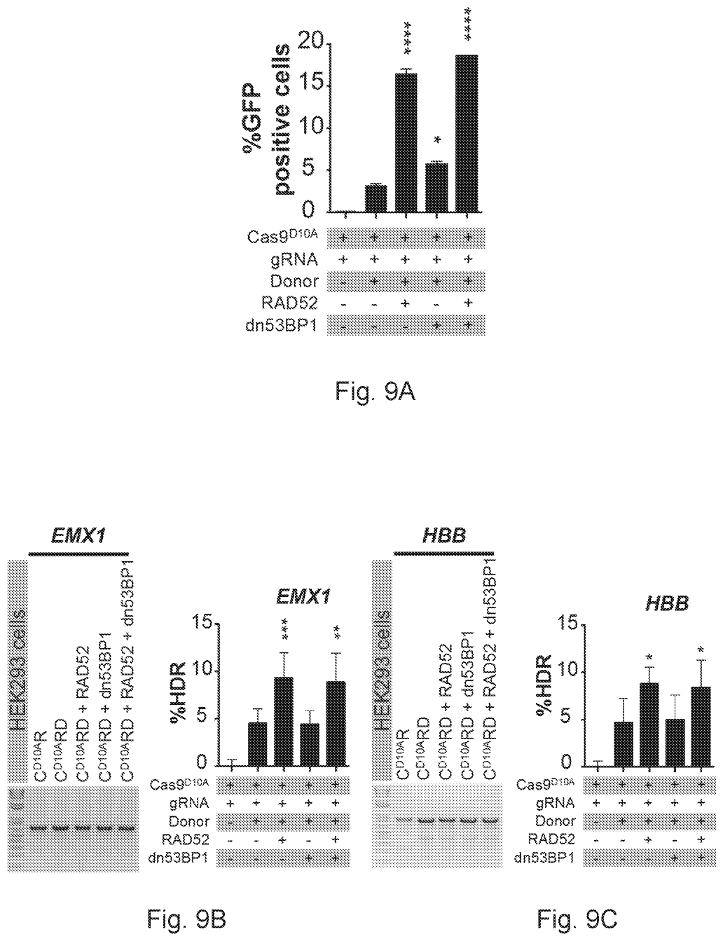

Co-expression of RAD52 and dn53BP1 increased the frequency of HDR in human cells at multiple loci. Similarly, using Cas9-nickase and targeting either the EMX1 or HBB locus, the methods described herein were demonstrated to increase HDR frequency (FIGS. 9A-9C).

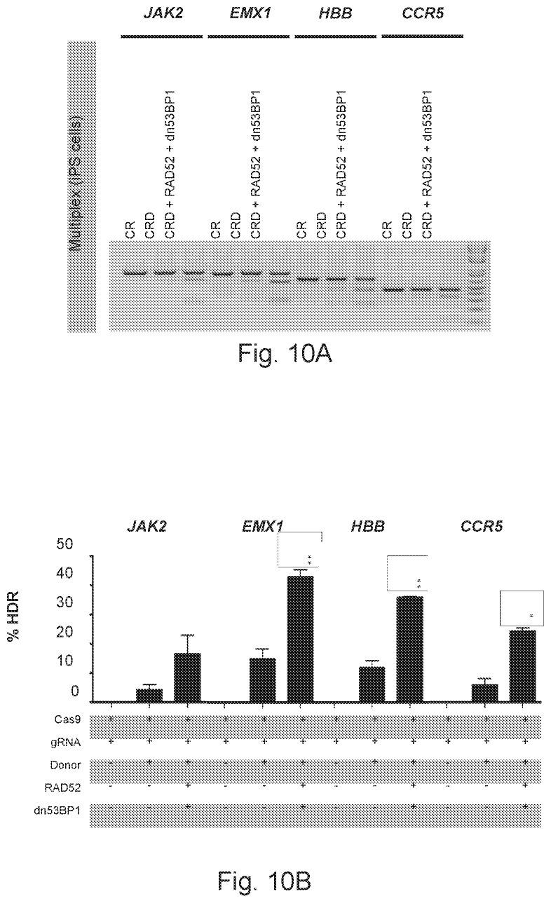

As demonstrated in FIGS. 10A-10B, it is possible to target multiple loci simultaneously by transfecting the cells with different gRNAs and donor templates. These results were demonstrated in iPS cells targeting four different loci at the same time (FIGS. 10A-10B).

1. A method of altering a target sequence of a target nucleic acid molecule, the method comprising contacting the target nucleic acid molecule with:
a. a nuclease;
b. at least one inhibitor of non-homologous end joining (NHEJ);
c. at least one agonist of homology-directed repair (HDR); and
d. a template nucleic acid.

2. The method of claim 1, wherein the inhibitor of NHEJ is selected from the group consisting of:
an inhibitor of Ku70; an inhibitor of Ku80; and an inhibitor of 53BP1.

3. (canceled)

4. The method of claim 1, wherein the agonist of HDR is selected from the group consisting of:
an agonist of RAD52; an agonist of RAD51; and an agonist of BLM.

5. The method of claim 1, wherein the inhibitor of NHEJ is an inhibitor of 53BP1 and the agonist of HDR is an agonist of Rad52.

6. (canceled)

7. (canceled)

8. A method of altering the sequence of a target nucleic acid molecule, the method comprising contacting the target nucleic acid molecule with:
a. a nuclease; and
b. at least one agonist of RAD52.

9. (canceled)

10. The method of claim 4, wherein the agonist of Rad52 is ectopic Rad52 polypeptide or a constitutively active RAD52 polypeptide.

11. The method of claim 4, wherein the agonist of RAD51 is ectopic RAD51 polypeptide or a constitutively active RAD51 polypeptide.

12. (canceled)

13. The method of claim 4, wherein the agonist of BLM is ectopic BLM polypeptide.

14. (canceled)

15. (canceled)

16. The method of claim 1, wherein the inhibitor of NHEJ is an inhibitor of Lig4.

17. The method of claim 16, wherein the inhibitor of Lig4 is SCR7.

18. The method of claim 1, wherein the target nucleic acid molecule is contacted with at least one agonist of HDR selected from E1B55K and E4orf6.

19. The method of claim 2, wherein the inhibitor of Ku70 is an inhibitory nucleic acid.

20. The method of claim 2, wherein the inhibitor of Ku80 is an inhibitory nucleic acid.

21. The method of claim 2, wherein the inhibitor of 53BP1 is an inhibitory nucleic acid or a dominant-negative 53BP1 (dn53BP1) polypeptide.

22. (canceled)

23. (canceled)

24. (canceled)

25. (canceled)

26. The method of claim 1, wherein the nuclease is a programmable nuclease or a meganuclease.

27. The method of claim 26, wherein the programmable nuclease is selected from the group consisting of:
Cas9; a Cas9 nickase mutant; TALEN; ZFNs; Cpf1; and SaCas9.

28.-44. (canceled)

45. The method of claim 1, wherein the contacting step occurs in a cell and further comprising contacting the cell with a cell cycle modulator.

46. The method of claim 45, wherein the cell cycle modulator increases the proportion of cells in late S or G2 phase.

47.-48. (canceled)

49. A composition comprising:
a) at least one inhibitor of non-homologous end joining (NHEJ); and/or
b) at least one agonist of homology-directed repair (HDR).

50.-69. (canceled)

70. A method of altering a target sequence of a target nucleic acid molecule, the method comprising contacting the target nucleic acid molecule with:
a. a Cas9 nuclease;
b. a guide RNA (gRNA) that can hybridize to a portion of the target nucleic acid molecule; and
c. a template nucleic acid;
wherein the ratio of the Cas9 nuclease:gRNA is 1:4 or greater.

71.-92. (canceled)


SEQUENCE LISTING (50 sequences)

SEQ ID NO: 1 - protein, 418 aa, Homo sapiens
MSGTEEAILGGRDSHPAAGGGSVLCFGQCQYTAEEYQAIQKALRQRLGPEYISSRMAGGGQKVCYIEGHRVINLANEMFGYNGWAHSITQQNVDFVDLNNGKFYVGVCAFVRVQLKDGSYHEDVGYGVSEGLKSKALSLEKARKEAVTDGLKRALRSFGNALGNCILDKDYLRSLNKLPRQLPLEVDLTKAKRQDLEPSVEEARYNSCRPNMALGHPQLQQVTSPSRPSHAVIPADQDCSSRSLSSSAVESEATHQRKLRQKQLQQQFRERMEKQQVRVSTPSAEKSEAAPPAPPVTHSTPVTVSEPLLEKDFLAGVTQELIKTLEDNSEKWAVTPDAGDGVVKPSSRADPAQTSDTLALNNQMVTQNRTPHSVCHQKPQAKSGSWDLQTYSADQRTTGNWESHRKSQDMKKRKYDPS

SEQ ID NO: 2 - protein, 226 aa, Homo sapiens
MSGTEEAILGGRDSHPAAGGGSVLCFGQCQYTAEEYQAIQKALRQRLGPEYISSRMAGGGQKVCYIEGHRVINLANEMFGYNGWAHSITQQNVDFVDLNNGKFYVGVCAFVRVQLKDGSYHEDVGYGVSEGLKSKALSLEKARKEAVTDGLKRALRLPLLGVSGRILYSLFSVHSVMCAGGLPTPTASAQTAPSSPCSSAVLRYAQEFWECTWKLYSGQRLPEITK

SEQ ID NO: 3 - protein, 341 aa, Homo sapiens
MFGYNGWAHSITQQNVDFVDLNNGKFYVGVCAFVRVQLKDGSYHEDVGYGVSEGLKSKALSLEKARKEAVTDGLKRALRSFGNALGNCILDKDYLRSLNKLPRQLPLEVDLTKAKRQDLEPSVEEARYNSCRPNMALGHPQLQQVTSPSRPSHAVIPADQDCSSRSLSSSAVESEATHQRKLRQKQLQQQFRERMEKQQVRVSTPSAEKSEAAPPAPPVTHSTPVTVSEPLLEKDFLAGVTQELIKTLEDNSEKWAVTPDAGDGVVKPSSRADPAQTSDTLALNNQMVTQNRTPHSVCHQKPQAKSGSWDLQTYSADQRTTGNWESHRKSQDMKKRKYDPS

SEQ ID NO: 4 - protein, 301 aa, Homo sapiens
MSGTEEAILGGRDSHPAAGGGSVLCFGQCQYTAEEYQAIQKALRQRLGPEYISSRMAGGGQKVCYIEGHRVINLANEMFGYNGWAHSITQQNVDFVDLNNGKFYVGVCAFVRVQLKDGSYHEDVGYGVSEGLKSKALSLEKARKEAVTDGLKRALRSFGNALGNCILDKDYLRSLNKLPRQLPLEVDLTKAKRQDLEPSVEEARYNSCRPNMALGHPQLQQVTSPSRPSHAVIPADQDCSSRSRGCCSGARPRRRPLMQKPELIRRGERGHAPAEAPAEAAAAAVPGADGEAAGSSLHAVS

SEQ ID NO: 5 - protein, 340 aa, Homo sapiens
MAMQMQLEANADTSVEEESFGPQPISRLEQCGINANDVKKLEEAGFHTVEAVAYAPKKELINIKGISEAKADKILTESRSVARLECNSVILVYCTLRLSGSSDSPASASRVVGTTGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 6 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 7 - protein, 280 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEIVSEERKRGNQNLQNLRLSLSS

SEQ ID NO: 8 - protein, 340 aa, Homo sapiens; identical to SEQ ID NO: 5

SEQ ID NO: 9 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 10 - protein, 1286 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 11 - protein, 1042 aa, Homo sapiens
MEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 12 - protein, 1977 aa, Homo sapiens
MPGEQMDPTGSQLDSDFSQQDTPCLIIEDSQPESQVLEDDSGSHFSMLSRHLPNLQTHKENPVLDVVSNPEQTAGEERGDGNSGFNEHLKENKVADPVDSSNLDTCGSISQVIEQLPQPNRTSSVLGMSVESAPAVEEEKGEELEQKEKEKEEDTSGNTTHSLGAEDTASSQLGFGVLELSQSQDVEENTVPYEVDKEQLQSVTTNSGYTRLSDVDANTAIKHEEQSNEDIPIAEQSSKDIPVTAQPSKDVHVVKEQNPPPARSEDMPFSPKASVAAMEAKEQLSAQELMESGLQIQKSPEPEVLSTQEDLFDQSNKTVSSDGCSTPSREEGGCSLASTPATTLHLLQLSGQRSLVQDSLSTNSSDLVAPSPDAFRSTPFIVPSSPTEQEGRQDKPMDTSVLSEEGGEPFQKKLQSGEPVELENPPLLPESTVSPQASTPISQSTPVFPPGSLPIPSQPQFSHDIFIPSPSLEEQSNDGKKDGDMHSSSLTVECSKTSEIEPKNSPEDLGLSLTGDSCKLMLSTSEYSQSPKMESLSSHRIDEDGENTQIEDTEPMSPVLNSKFVPAENDSILMNPAQDGEVQLSQNDDKTKGDDTDTRDDISILATGCKGREETVAEDVCIDLTCDSGSQAVPSPATRSEALSSVLDQEEAMEIKEHHPEEGSSGSEVEEIPETPCESQGEELKEENMESVPLHLSLTETQSQGLCLQKEMPKKECSEAMEVETSVISIDSPQKLAILDQELEHKEQEAWEEATSEDSSVVIVDVKEPSPRVDVSCEPLEGVEKCSDSQSWEDIAPEIEPCAENRLDTKEEKSVEYEGDLKSGTAETEPVEQDSSQPSLPLVRADDPLRLDQELQQPQTQEKTSNSLTEDSKMANAKQLSSDAEAQKLGKPSAHASQSFCESSSETPFHFTLPKEGDIIPPLTGATPPLIGHLKLEPKRHSTPIGISNYPESTIATSDVMSESMVETHDPILGSGKGDSGAAPDVDDKLCLRMKLVSPETEASEESLQFNLEKPATGERKNGSTAVAESVASPQKTMSVLSCICEARQENEARSEDPPTTPIRGNLLHFPSSQGEEEKEKLEGDHTIRQSQQPMKPISPVKDPVSPASQKMVIQGPSSPQGEAMVTDVLEDQKEGRSTNKENPSKALIERPSQNNIGIQTMECSLRVPETVSAATQTIKNVCEQGTSTVDQNFGKQDATVQTERGSGEKPVSAPGDDTESLHSQGEEEFDMPQPPHGHVLHRHMRTIREVRTLVTRVITDVYYVDGTEVERKVTEETEEPIVECQECETEVSPSQTGGSSGDLGDISSFSSKASSLHRTSSGTSLSAMHSSGSSGKGAGPLRGKTSGTEPADFALPSSRGGPGKLSPRKGVSQTGTPVCEEDGDAGLGIRQGGKAPVTPRGRGRRGRPPSRTTGTRETAVPGPLGIEDISPNLSPDDKSFSRVVPRVPDSTRRTDVGAGALRRSDSPEIPFQAAAGPSDGLDASSPGNSFVGLRVVAKWSSNGYFYSGKITRDVGAGKYKLLFDDGYECDVLGKDILLCDPIPLDTEVTALSEDEYFSAGVVKGHRKESGELYYSIEKEGQRKWYKRMAVILSLEQGNRLREQYGLGPYEAVTPLTKAADISLDNLVEGKRKRRSNVSSPATPTASSSSSTTPTRKITESPRASMGVLSGKRKLITSEEERSPAKRGRKSATVKPGAVGAGEFVSPCESGDNTGEPSALEEQRGPLPLNKTLFLGYAFLLTMATTSDKLASRSKLPDGPTGSSEEEEEFLEIPPFNKQYTESQLRAGAGYILEDFNEAQCNTAYQCLLIADQHCRTRKYFLCLASGIPCVSHVWVHDSCHANQLQNYRNYLLPAGYSLEEQRILDWQPRENPFQNLKVLLVSDQQQNFLELWSEILMTGGAASVKQHHSSAHNKDIALGVFDVVVTDPSCPASVLKCAEALQLPVVSQEWVIQCLIVGERIGFKQHPKYKHDYVSH

SEQ ID NO: 13 - protein, 9 aa, Unknown
  Description of Unknown Homing endonuclease peptide
LAGLIDADG

SEQ ID NO: 14 - DNA, 19 bp, Artificial Sequence
  Description of Artificial Sequence Synthetic oligonucleotide
gggccactagggacaggat

SEQ ID NO: 15 - DNA, 184 bp, Artificial Sequence
  Description of Artificial Sequence Synthetic polynucleotide
tgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggca

SEQ ID NO: 16 - DNA, 20 bp, Artificial Sequence
  Description of Artificial Sequence Synthetic oligonucleotide
aattatggagtatgtgtctg

SEQ ID NO: 17 - DNA, 22 bp, Artificial Sequence
  Description of Artificial Sequence Synthetic primer
acgttgatggcagttgcaggtc

SEQ ID NO: 18 - DNA, 27 bp, Artificial Sequence
  Description of Artificial Sequence Synthetic primer
ctgacagagttgctagacactgggttg

SEQ ID NO: 19 - DNA, 184 bp, Artificial Sequence
  Description of Artificial Sequence Synthetic polynucleotide
ttccttagtctttctttgaagcagcaagtatgatgagcaagctttctcacaagcatttggttttaaattatggagtatgtgtgtttaaacctgtggagacgagagtaagtaaaactacaggctttctaatgcctttctcagagcatctgtttttgtttatatagaaaattcagtttcaggatca

SEQ ID NO: 20 - DNA, 20 bp, Artificial Sequence
  Description of Artificial Sequence Synthetic oligonucleotide
gtcacctccaatgactaggg

SEQ ID NO: 21 - DNA, 24 bp, Artificial Sequence
  Description of Artificial Sequence Synthetic primer
gtcttcccatcaggctctcagctc

SEQ ID NO: 22 - DNA, 23 bp, Artificial Sequence
  Description of Artificial Sequence Synthetic primer
gagctggaggtagagaccagggt

SEQ ID NO: 23 - DNA, 184 bp, Artificial Sequence
  Description of Artificial Sequence Synthetic polynucleotide
aagaagggctcccatcacatcaaccggtggcgcattgccacgaagcaggccaatggggaggacatcgatgtcacctccaatgactagtttaaacgggtgggcaaccacaaacccacgagggcagagtgctgcttgctgctggccaggcccctgcgtgggcccaagctggactctggccactccc

SEQ ID NO: 24 - DNA, 20 bp, Artificial Sequence
  Description of Artificial Sequence Synthetic oligonucleotide
agtctgccgttactgccctg

SEQ ID NO: 25 - DNA, 24 bp, Artificial Sequence
  Description of Artificial Sequence Synthetic primer
tgccagaagagccaaggacaggta

SEQ ID NO: 26 - DNA, 24 bp, Artificial Sequence
  Description of Artificial Sequence Synthetic primer
catcaagcgtcccatagactcacc

SEQ ID NO: 27 - DNA, 188 bp, Artificial Sequence
  Description of Artificial Sequence Synthetic polynucleotide
atttgcttctgacacaactgtgttcactagcaacctcaaacagacaccatggtgcatctgactcctgaggagaagtctgccgttactgccgtttaaacctgtggggcaaggtgaacgtggatgaagttggtggtgaggccctgggcaggttggtatcaaggttacaagacaggtttaaggagaccaat

SEQ ID NO: 28 - DNA, 20 bp, Artificial Sequence
  Description of Artificial Sequence Synthetic oligonucleotide
tcactatgctgccgcccagt

SEQ ID NO: 29 - DNA, 20 bp, Artificial Sequence
  Description of Artificial Sequence Synthetic primer
ctgcaaaaggctgaagagca

SEQ ID NO: 30 - DNA, 25 bp, Artificial Sequence
  Description of Artificial Sequence Synthetic primer
ccccaagatgactatctttaatgtc

SEQ ID NO: 31 - DNA, 184 bp, Artificial Sequence
  Description of Artificial Sequence Synthetic polynucleotide
catgactgacatctacctgctcaacctggccatctctgacctgtttttccttcttactgtccccttctgggctcactatgctgccgccgtttaaaccagtgggactttggaaatacaatgtgtcaactcttgacagggctctattttataggcttcttctctggaatcttcttcatcatcctcc

SEQ ID NO: 32 - DNA, 20 bp, Artificial Sequence
  Description of Artificial Sequence Synthetic oligonucleotide
gctgtgtttgcgtctctccc

SEQ ID NO: 33 - DNA, 192 bp, Artificial Sequence
  Description of Artificial Sequence Synthetic polynucleotide
gtccatgctgtgtttgctttaaaagccaggacggtcacctttggggtggtgacaagtgtgatcacttgggtggtggctgtgtttgcgtctctgtttaaaccccaggaatcatctttaccagatctcaaaaagaaggtcttcattacacctgcagctctcattttccatacagtcagtatcaattctggaaga

SEQ ID NO: 34 - DNA, 20 bp, Artificial Sequence
  Description of Artificial Sequence Synthetic oligonucleotide
gctactctctctttctggcc

SEQ ID NO: 35 - DNA, 20 bp, Artificial Sequence
  Description of Artificial Sequence Synthetic oligonucleotide
tgatcttggctactatacca

SEQ ID NO: 36 - DNA, 21 bp, Artificial Sequence
  Description of Artificial Sequence Synthetic primer
gagctgcaagcctgttatgtg

SEQ ID NO: 37 - DNA, 20 bp, Artificial Sequence
  Description of Artificial Sequence Synthetic primer
cgcaacccagtaccacttac

SEQ ID NO: 38 - DNA, 184 bp, Artificial Sequence
  Description of Artificial Sequence Synthetic polynucleotide
gaattggggctcatcaattatcaattctttcacccttcaaataattcttttctttattcaatgcctgtagctattgcattaatgaccacagcggtcatctctacctgcgaccatggtatagtagccaagatcaagagagtgatcatggagagagacacttaccctcggaagtggggtttaggtc

SEQ ID NO: 39 - DNA, 51 bp, Homo sapiens
ccacagcggtcatctctacctgcgaccatggtatagtagccaagatcaaga

SEQ ID NO: 40 - DNA, 51 bp, Artificial Sequence
  Description of Artificial Sequence Synthetic oligonucleotide
ccacagtggtcatctctacctgcgaccatggtatagtagccaagatcaaga

SEQ ID NO: 41 - DNA, 51 bp, Artificial Sequence
  Description of Artificial Sequence Synthetic oligonucleotide
ccacagcggtcatctctacctgcgatcatggtatagtagccaagatcaaga

SEQ ID NO: 42 - DNA, 23 bp, Homo sapiens
tcactatgctgccgcccagtggg

SEQ ID NO: 43 - DNA, 23 bp, Homo sapiens
gctgtgtttgcgtctctcccagg

SEQ ID NO: 44 - DNA, 23 bp, Homo sapiens
gctgtgtccgcgcctctccccgg

SEQ ID NO: 45 - DNA, 23 bp, Homo sapiens
gctgtgtttgcgcctttcccagg

SEQ ID NO: 46 - DNA, 23 bp, Homo sapiens
gctgtgtttgtgcctcccccagg

SEQ ID NO: 47 - DNA, 23 bp, Homo sapiens
gctgtgctagcacctcccccagg

SEQ ID NO: 48 - DNA, 23 bp, Homo sapiens
gctgtgtgctcgtctctccctgg

SEQ ID NO: 49 - DNA, 23 bp, Homo sapiens
tgggtgtctgcagctctcccagg

SEQ ID NO: 50 - DNA, 23 bp, Homo sapiens
cacgagtgtgcgcctcccccagg